(19) United States
(12) Patent Application Publication
(10) Pub. No.: US 2009/0185973 A1
(43) Pub. Date: Jul. 23, 2009

(54) METHODS, COMPOSITIONS, UNIT DOSAGE FORMS, AND KITS FOR PHARMACOLOGIC STRESS TESTING WITH REDUCED SIDE EFFECTS

(75) Inventor: Philippe Gorny, Paris (FR)

Correspondence Address: DECHERT LLP, P.O. BOX 390460, MOUNTAIN VIEW, CA, 94039-0460, US

(73) Assignee: ADENOBIO N.V., Amsterdam Schiphol (NL)

(21) Appl. No.: 12/351,227

(22) Filed: Jan. 9, 2009

Related U.S. Application Data
(60) Provisional application No. 61/085,791, filed on Aug. 1, 2008.
(60) Provisional application No. 61/022,695, filed on Jan. 22, 2008.

(51) Int. Cl. A61K 51/00; A61K 31/7076; C07D 487/02; A61K 49/04; A61K 49/06; A61K 49/00; A61K 49/22; B42D 15/00

(52) U.S. Cl. 424/1.11; 514/46; 544/256; 424/9.4; 424/9.3; 424/9.1; 283/81

(57) ABSTRACT

Methods are presented for concurrent or sequential administration of pharmaceutical compositions of adenosine, dipyridamole, or combinations thereof, at dosages below the respective single agent doses. Methods are provided for detecting the presence and/or assessing the severity of myocardial ischemia during pharmacologic stress tests. Methods include sequential administration of a dipyridamole bolus followed by intravenous infusion of adenosine and concurrent administration of adenosine and dipyridamole with or without dipyridamole pretreatment. The methods are useful for exploiting the vasodilating abilities of adenosine at doses at which side effects related to adenosine are substantially reduced while optimal coronary artery perfusion is achieved. Also presented are compositions, unit dosage forms, and kits that are useful in performing the methods.

### CROSS-REFERENCE TO RELATED APPLICATIONS

This application claims benefit under 35 U.S.C. §119(e) to U.S. provisional application Ser. No. 61/085,791, filed 1 Aug. 2008, and U.S. provisional application Ser. No. 61/022,695, filed 22 Jan. 2008, the disclosures of which are incorporated herein by reference in their entireties.

### 1. BACKGROUND

Functional assessment of myocardium, in particular the evaluation of the myocardium's oxygen status, is important in guiding therapeutic decisions in the care of patients with cardiac ischemia. In current clinical practice, myocardial ischemia status is most often assessed using non-invasive nuclear perfusion imaging methodologies, such as planar scintigraphy or single photon emission computed tomography (SPECT), with thallium and technetium as the most frequently used isotopes. Presently, other types of imaging that entail less exposure to radiation and/or provide improved visualization of myocardium are becoming available. Positron emission tomography (PET) with rubidium-82, ³N-ammonia (¹³NH₃), or ¹⁵O-labeled water (H₂¹⁵O), has been gaining recognition as providing improved images with less radiation. Another technology, myocardial contrast echography (MCE), uses agents detectable by ultrasound to study myocardium perfusion and heart function in real time with a single test. Doppler echography, either semi-invasive transesophageal doppler echography or non-invasive transthoracic doppler echocardiography, allows examination of the motion of ventricular walls and measurement of coronary flow reserve. Yet another imaging technology, X-ray computed tomography (CT) scanning, has been used in studying myocardial perfusion possibly coupled with coronary angiograms. High speed CT scanners, such as ultrafast CT scanners, are capable of taking multiple images of the heart within the time of a single heartbeat. Recently, this technology has been further improved using dual-energy imaging leading to the development of ultra high speed CT scanners, such as the Somatom Definition Flash, capable of scanning an entire human thorax in 0.6 seconds, and heart in 250 milliseconds, thereby reducing radiation exposure and permitting repeated scanning under various conditions for myocardial perfusion imaging (MPI) studies.

Each of these functional tests typically require that the patient's heart be “stressed” in order to assess cardiac function. Such stress can be induced either through controlled exercise or by pharmacologic means. These tests are commonly referred to as “stress tests”. Pharmacological stressors for functional assessment of myocardium act through coronary vasodilation: by dilating normal vessels to a greater extent than diseased vessels, these agents establish a shunt, or “myocardial steal”, that produces differential increases in blood flow in healthy vs. diseased arteries in patients with coronary artery disease, optimizing the discriminatory imaging of cardiac muscle areas in need of oxygen supply.

Adenosine and dipyridamole are coronary vasodilators, each of which is separately approved for individual use as a pharmacologic stressor for stress testing. Adenosine acts directly by stimulating adenosine purinergic P1 receptors on the arterial wall. Dipyridamole is believed to work indirectly by blocking reuptake of adenosine at the cellular level, leading to an increase in endogenous adenosine concentration in the blood. Dipyridamole produces similar near-maximal coronary hyperemia to that produced by exogenous adenosine, but less quickly.

To ensure near-maximal coronary vasodilation, and to provide sufficient time for the acquisition of cardiac images, adenosine is infused for 6 minutes at a dosage rate of 140 μg/kg patient body weight/min; dipyridamole is infused for 4 minutes at 140 μg/kg patient body weight/min. Thus, the total recommended dose of adenosine is 0.84 mg/kg, and the total recommended dose for dipyridamole is 0.56 mg/kg at the minimum and 0.80 mg/kg on average in a 4 minute infusion. If vasodilation is insufficient, the total dose of dipyridamole can be increased up to 0.95 mg/kg, administered over a 6 minute infusion.

Although infused for only a few minutes, compounds that stimulate adenosine receptors are accompanied by numerous uncomfortable adverse effects. With adenosine, the most frequently reported are flushing (44%), chest pain or chest discomfort (40%), dyspnea (28%), headache (18%), throat or neck or jaw discomfort (15%), and gastrointestinal discomfort (13%); other side effects (e.g., atrioventricular blocks) are less frequent.

The adverse effects of adenosine are dose-dependent. Symptoms such as heat sensation, flushed face, dyspnea and chest pain increase as adenosine dosage is increased from 60 to 140 μg/kg/min, in a six minute infusion. Chest pain typically appears at doses of 90 μg/kg/min, and becomes frequent at 120 μg/kg/min. At a dosage of 70 μg/kg/min or less, it has been noted that adenosine adverse reactions are very few and of mild intensity. However, when administered by intravenous perfusion at 70 μg/kg/min or less, or even at 90-120 μg/kg/min, adenosine shows reduced efficacy, and is not recommended for stress testing at such reduced dosages.

The side effect profile of dipyridamole is similar, but with adverse events occurring less often. However, dipyridamole side effects last longer, are more difficult to manage, and thus more frequently require extra patient monitoring time and the administration of intravenous aminophylline as an antidote.

Because dipyridamole is understood to act by increasing endogenous adenosine, use of both adenosine and dipyridamole at full intravenous dosage is contraindicated. Similarly, oral intake of dipyridamole prior to an adenosine pharmacologic stress testing is generally avoided.

In an effort to reduce side effects at maximally effective agonist doses, adenosinergic agents are being developed that are selective for the A2a receptor subtype. See, e.g., U.S. Pat. Nos. 6,531,457; 6,448,235; 6,322,771; and 5,877,180. Specific compounds either recently approved by FDA or still in development include regadenoson, binodenoson and apadenoson. However, despite increased receptor selectivity, these adenosinergic agents also exhibit side effects unrelated to activity on the A2a receptor, due to their incomplete selectivity. The overall reduction of side effects remains modest and sometimes, as is the case for regadenoson, show an increase in frequency and severity of side effects. Regadenoson treatment, for example, results in an increase in dyspnea, headache and gastrointestinal disorders as compared to treatment with the reference drug Adenoscan (adenosine). These compounds also have a longer duration of action than adenosine (e.g 10±6 minutes for binodenoson) due to a tighter affinity to A2a receptor. Accordingly, the A2a-related side effects, e.g., flushing, headache, and dyspnea, are longer lasting. Thus, although more specific than adenosine, these agents may be more likely to trigger prolonged side effects requiring administration of pharmacologic antidotes, than adenosine itself, whose side effects rapidly dissipate once administration is stopped. Additionally, these products (e.g., Regadenoson) may induce direct sympathetic stimulation, in particular an increase in heart rate that is greater than that observed with adenosine. Therefore the potential risk of ventricular arrhythmia in severe coronary patients should not be underestimated and could pose safety problems in the future.

There thus exists a continuing need in the art for injectable agents that can be used for pharmacologic stress testing, that have the rapid onset and short half-life of adenosine, and thus can be managed clinically in the same manner as adenosine, and that provide maximal efficacy with reduced side effects.

### 2. SUMMARY

Dipyridamole is believed to act indirectly by increasing endogenous adenosine concentration. In clinical practice, the compound has a side effect profile similar to that of adenosine, and is also known to potentiate exogenous adenosine adverse events to such an extent that its combination with adjunctively administered adenosine is normally contraindicated (“drugs that augment the effects of adenosine should be withheld for at least five half-lives prior to the use of Adenoscan”, quoted from the US FDA-approved Adenoscan drug label). However, I have discovered that extremely low parenteral doses of dipyridamole—on the order of 5% of the dose now used clinically in cardiac imaging studies—can surprisingly ensure optimal vasodilation effects of adjunctively administered adenosine without commensurate potentiation of adenosine's side effects. See commonly assigned U.S. patent application Ser. No. 11/772,784, and international application no. PCT/EP2007/005923, published as WO 2008/003479, the disclosures which are incorporated herein by reference in their entireties. This allows use of adenosine at reduced dosages to effect coronary vasodilation, e.g., for functional assessment of myocardial function, and provides equal or superior efficacy as compared to current protocols while reducing side effects. Moreover, the clinical and hemodynamic effects advantageously stop less than one minute after cessation of adenosine administration.

Accordingly, described herein are methods, compositions, unit dosage forms and kits that use subclinical doses of dipyridamole, lacking significant hemodynamic and clinical effect when used alone, to modulate effects of adjunctively administered adenosine, (i) permitting reduction in the exogenous adenosine administered dose, and (ii) reducing related adverse events incidence and severity, while maintaining the same efficacy and duration of action as adenosine at its recommended single agent dosage.

In one aspect, methods are provided for effecting coronary vasodilation for cardiac diagnosis.

In various embodiments, the methods comprise: (i) parenterally administering an adenosine modulator, such as dipyridamole; and (ii) sequentially thereafter parenterally administering an adenosine receptor agonist, such as adenosine. Each of dipyridamole and the adenosine receptor agonist is administered at a dosage lower than that required for maximal coronary vasodilation when administered as a single agent by identical parenteral route.

In some embodiments, the adenosine receptor agonist is selected from: adenosine, adenosine triphosphate (ATP), adenosine diphosphate (ADP), adenosine monophosphate (AMP), and pro-drugs and pharmaceutically acceptable salts of adenosine or AMP, ADP, ATP. Whatever the identity of the adenosine receptor agonist, the ratio between adenosine or the adenosine agonist and dipyridamole is about 2:1 to 10:1 and preferably 7:1.

Each route of parenteral administration may be independently selected from: intra-arterial, intravenous, and atrial administration.

In some embodiments, dipyridamole is administered by intravenous or intra-arterial bolus injection. In certain embodiments, dipyridamole is administered as an intravenous or intra-arterial bolus at a dosage of no more than about 140 μg/kg, no more than about 50 μg/kg, even no more than about 40 μg/kg, and typically at a dosage of at least about 14 μg/kg. For example, in some embodiments, dipyridamole is administered as an intravenous or intra-arterial bolus at a dosage of about 23 to about 60 μg/kg, such as about 35 μg/kg or about 40 μg/kg which is the preferred dosage.

In some embodiments dipyridamole is administered as an intravenous bolus immediately followed by the intravenous infusion of adenosine. Immediately means as soon as clinically practicable, typically within about 2 to 30 seconds.

In various embodiments dipyridamole is administered as a 5 to 30 second bolus prior to adenosine infusion of about 1 to about 6 minutes.

In a variety of embodiments, the adenosine receptor agonist is administered intravenously over about 2 or about 3 minutes following dipyridamole bolus injection.

In some embodiments, dipyridamole is administered by intravenous infusion over about 1 or about 4 minutes.

In some embodiments, the adenosine receptor agonist administration is begun after completion of dipyridamole administration, such as between about 30 seconds and about 2 minutes after dipyridamole injection or infusion.

In typical embodiments, the adenosine receptor agonist is adenosine, administered by intravenous infusion at a dosage rate of about 35 μg/kg/min-100 μg/kg/min. In these embodiments, adenosine is administered at a dosage rate of no more than about 100 μg/kg/min. In some embodiments, adenosine is administered at a dosage rate of no more than about 70 μg/kg/min, even no more than about 50 μg/kg/min. In exemplary embodiments, the adenosine receptor agonist is adenosine, administered by intravenous infusion at a dosage rate of at least about 35 μg/kg/min, even at least about 50 μg/kg/min. For example, in some embodiments, adenosine is administered by intravenous infusion at a rate of about 50 μg/kg/min to about 70 μg/kg/min which is the preferred dosage.

In certain embodiments, the adenosine receptor agonist is adenosine, the total dose of dipyridamole is 23 to 40 μg/kg, and the dosage rate for adenosine is 50 to 70 μg/kg/min. For example, in some embodiments, the total dose of dipyridamole is 40 μg/kg and the dosage rate for adenosine is 70 μg/kg/min.

In a variety of embodiments, adenosine and dipyridamole are administered by intravenous infusion. In some embodiments, adenosine is administered into a coronary artery at a dose of about 10 to about 20 μg/min, regardless of patient weight, after intravenous bolus injection of dipyridamole at a dose of about 20 to about 40 μg/kg, preferably 40 μg/kg.

In certain embodiments, the method usefully comprises the step of assessing cardiac function. Assessing cardiac function may include use of one or more techniques selected from: electrocardiography, M mode echography, two dimensional echography, three dimensional echography, echo-doppler (e.g., Transthoracic echo-doppler), cardiac imaging, planar (conventional) scintigraphy, single photon emission computed tomography (SPECT), dynamic single photon emission computed tomography, positron emission tomography (PET), first pass radionuclide angiography, equilibrium radionuclide angiography, nuclear magnetic resonance (NMR) imaging, myocardial perfusion contrast echocardiography (MCE or real-time MCE), digital subtraction angiography (DSA), x-ray computed tomography (CINE CT), including high speed and ultra high speed CT scanning. In certain embodiments, functional assessment is specifically performed by SPECT; in other embodiments, the assessment is specifically performed by PET. In other embodiments the assessment is specifically performed by MCE. In other embodiments it is specifically performed by CT scan, including high speed and ultra high speed CT scan.

In some embodiments, assessing cardiac function includes parenteral administration of an isotope, and the isotope is administered no less than 1 minute but before 2.75 minutes. In some embodiments the isotope is delivered after 1.1 minute, after 1.2 minute, after 1.3 minute, after 1.4 minute, after 1.5 minute, after 1.6 minute, after 1.7 minute, after 1.8 minute, after 1.9 minute, after 2 minutes (which is the preferred method), after 2.1 minutes, after 2.2 minutes, after 2.3 minutes, after 2.4 minutes, after 2.5 minutes and always before 2.75 minutes, when dipyridamole and the adenosine receptor agonist are administered sequentially.

In some embodiments, the method may comprise the step of assessing cardiac function using the injection or formation of contrast agents detected by ultrasound techniques in a period of time ranging from 10 seconds to 3 minutes. These techniques may include parenteral administration of an agent such as, hydrophobic drugs and/or polymers (e.g polyethylene glycol), in the form of microspheres or nanospheres and/or microbubbles made of gas including air or compositions comprising combinations of these agents (e.g. perflubutane polymers)

In one aspect, methods of effecting coronary vasodilation for cardiac diagnosis are provided. The methods comprise concurrently administering an adenosine receptor agonist, such as adenosine, and an adenosine modulator, such as dipyridamole. In various embodiments, adenosine and dipyridamole are administered parenterally at an adenosine:dipyridamole weight ratio of about 2:1 to about 10:1.

In certain embodiments, a small fraction of the total dipyridamole dose is used as a priming dose—acting as a pretreatment—preceding the concurrent administration of dipyridamole with adenosine.

In certain embodiments, the adenosine:dipyridamole ratio is about 2:1 to about 4:1, for example 4:1, or about 8:1, or preferably 7:1.

In various embodiments, adenosine is administered at a dosage rate of about 35 to 100 μg/kg/min and dipyridamole is administered at a dosage rate of about 3.5-50 μg/kg/min. In some embodiments, adenosine is administered at a dosage rate of about 70 μg/kg/min and dipyridamole is administered at a dosage rate of about 10 μg/kg/min (which is the preferred combination dose); adenosine is administered at a dosage rate of about 70 μg/kg/min and dipyridamole is administered at a dosage rate of about 8.75 μg/kg/min; adenosine is administered at a dosage rate of about 50 μg/kg/min and dipyridamole is administered at a dosage rate of about 12.5-25 μg/kg/min.

Adenosine and dipyridamole may be parenterally administered continuously for a period of at least about 1 minute, typically less than about 6 minutes. In certain embodiments, adenosine and dipyridamole are parenterally administered continuously for a period of about 3 or 4 minutes. In other embodiments for a period of about 1 to 2 minutes.

In some embodiments of the methods presented herein, adenosine and dipyridamole are administered as a single composition. In some embodiments, adenosine and dipyridamole are administered concurrently from separate compositions. In certain embodiments, two modes are combined (e.g. a dipyridamole priming dose is administered using a dipyridamole unit dosage form, followed by administration of a unit dose of a combined composition).

In certain embodiments, the method usefully comprises the step of assessing cardiac function. Assessing cardiac function may include use of one or more techniques selected from: electrocardiography, M mode echography, two dimensional echography, three dimensional echography, echo-doppler (e.g., Transthoracic echo-doppler), cardiac imaging, planar (conventional) scintigraphy, single photon emission computed tomography (SPECT), dynamic single photon emission computed tomography, positron emission tomography (PET), first pass radionuclide angiography, equilibrium radionuclide angiography, nuclear magnetic resonance (NMR) imaging, myocardial perfusion contrast echocardiography (also called real-time MCE), digital subtraction angiography (DSA), x-ray computed tomography (CINE CT), including high speed and ultra high speed CT scanning. In certain embodiments, functional assessment is specifically performed by SPECT; in other embodiments, the assessment is specifically performed by PET. In other embodiments the assessment is specifically performed by MCE. In other embodiments it is specifically performed by CT scan, including high speed and ultra high speed CT scan.

In certain embodiments of the methods that comprise assessment of cardiac function, assessing cardiac function includes parenteral administration of an isotope. The isotope is typically administered no less than about 1 minute and no more than about 3 minutes after the concurrent parenteral administration of adenosine and dipyridamole has begun. In some embodiments isotope can be administered no less than 1.1 minute, no less than 1.2 minute, no less than 1.3 minute, no less than 1.4 minute, no less than 1.5 minute, no less than 1.6 minute, no less than 1.7 minute, no less than 1.8 minute, no less than 1.9 minute, no less than 2 minutes, no less than 2.1 minutes, no less than 2.2 minutes, no less than 2.3 minutes, no less than 2.4 minutes, no less than 2.5 minutes, no less than 2.6 minutes, no less than 2.7 minutes, no less than 2.8 minutes, no less than 2.9 minutes after the concurrent administration of adenosine and dipyridamole has begun. In other embodiments, where a fraction of the dipyridamole total dose (priming dose) is administered prior to the concurrent administration mode, the isotope is injected after only 2 minutes and always before 2.5 minutes.

In other embodiments, the methods comprise assessing myocardial perfusion and function by transthoracic doppler-echography or myocardial perfusion contrast echography; myocardial perfusion contrast echocardiography (real-time MCE) may include parenteral administration of an agent detectable by ultrasound techniques such as, but not only, hydrophobic drugs and/or polymers (e.g polyethylene glycol), in the form of microspheres or nanospheres and/or microbubbles made of gas including air or compositions comprising combinations of these agents (e.g. perflubutane polymers).

In some aspects, pharmaceutical compositions comprising adenosine and dipyridamole are presented. The compositions comprise adenosine and dipyridamole in adenosine:dipyridamole weight ratios of about 2:1 to about 10:1, such as about 2:1 to about 4:1. In some embodiments, the ratio is about 8:1 and preferably 7:1.

In various embodiments, adenosine and dipyridamole are present in amounts that permit adenosine to be administered at a dosage rate of about 35 to about 100 μg/kg/min and dipyridamole to be administered at a dosage rate of about 3.5 to about 50 μg/kg/min.

In various embodiments adenosine is administered at a dosage rate of about 70 μg/kg/min and dipyridamole at a dosage rate of about 10 μg/kg/min.

In various embodiments, the composition may be a sterile fluid, such as a sterile fluid suitable for parenteral administration, such as intravenous administration. Its pH is generally acid (2 to 4). In some embodiments, adenosine and dipyridamole are present at concentrations that permit direct intravenous administration and immediate dilution into the venous line.

In various embodiments, adenosine and dipyridamole are present at concentrations that permit administration of adenosine at a dosage rate of about 70 μg/kg/min and dipyridamole at a dosage rate of about 8.75 to about 10 μg/kg/min. In some embodiments, adenosine and dipyridamole are present at concentrations that permit administration of adenosine at a dosage rate of about 50 μg/kg/min and dipyridamole at a dosage rate of about 12.5 to about 25 μg/kg/min.

In a range of embodiments of the pharmaceutical compositions here provided, the concentration of adenosine is about 1 to 10 mg/ml. Usefully, the concentration of adenosine is about 3 mg/ml or 4 mg/ml, even 5 mg/ml, or 7 mg/ml.

In certain embodiments, the concentration of dipyridamole is about 0.1 to 4 mg/ml, such as: 3 mg/7 ml and 6 mg/14 ml (0.43 mg/ml), 4 mg/7 ml and 8 mg/14 ml (0.57 mg/ml), 5 mg/7 ml (0.71 mg/ml), 6 mg/7 ml (0.85 mg/ml) or 1 mg/ml as shown in Table 1, hereinbelow.

In various embodiments of pharmaceutical compositions comprising adenosine, either as sole active, or in combination with dipyridamole, the composition has a pH less than about 4.5, less than about 4.4, less than about 4.3, less than about 4.2, less than about 4.1, less than about 4.0, less than about 3.9, less than about 3.8, less than about 3.7, even less than about 3.6, including intermediate nonintegral pH values there between. In certain embodiments, the pH is between about 2 and 4.

In one aspect, unit dosage forms are provided that contain pharmaceutical compositions as above-described, comprising adenosine and dipyridamole.

In some embodiments, the unit dosage form contains about 2 to 50 ml of the pharmaceutical composition formulated as a sterile fluid, typically a sterile, nonpyrogenic, solution suitable for parenteral administration. In some embodiments, the unit dosage form contains about 2 ml, 3 ml, 4 ml, 5 ml, 6 ml, 7 ml, 8 ml or 14 ml (see Table 1).

In some embodiments, the unit dosage form contains about 5 to 60 mg of adenosine and about 0.5 to 30 mg of dipyridamole; the composition is a solid capable of sterile reconstitution in a physiologically acceptable solvent or solution.

In exemplary embodiments, the unit dosage form contains about 14 mg of adenosine and about 2 mg of dipyridamole, about 21 mg of adenosine and about 3 mg of dipyridamole; about 28 mg of adenosine and about 4 mg of dipyridamole; about 35 mg of adenosine and about 5 mg of dipyridamole; about 42 mg of adenosine and about 6 mg of dipyridamole; about 56 mg of adenosine and about 8 mg of dipyridamole.

In some embodiments, the unit dosage forms are usefully prefilled syringes.

In some embodiments the unit dosage forms are vials with compositions that can be sampled using empty syringes of standard 10, 15, 20, and even 30 ml total capacity. In various embodiments, the barrel of the syringe—whether prefilled or not—is usefully labeled with weight (e.g., kilograms or pounds) graduation marks so as to facilitate weight-adjusted dosing of the active.

In some embodiments, the weight graduation scale is in kilograms (kilo or kg). In certain embodiments, kilo graduation scales range from 10 or 40 kilograms up to 100 or 125 or 133 or 150 or 166 or even 200 kilograms, depending on the syringe capacity. Milliliter per kilogram equivalence can range typically from 0.01 ml=1 kg to 0.2 ml=1 kg depending on the dose, the volume of liquid in the syringe, and the recommended infusion time. In some embodiments, the graduation scale is such that a one kilogram interval can equal 0.03 ml, or 0.04 ml or 0.042 ml, or 0.0525 ml, or 0.056 ml, or 0.07 ml, according to needs and the features of the syringe used. In some embodiments, syringes are provided with a weight graduation scale in pounds.

In another aspect, unit dosage forms of dipyridamole are provided. In various embodiments, dipyridamole is provided in solution at a concentration of about 0.1 to 4 mg/ml.

In certain embodiments, the dipyridamole concentration is usefully about 3 to 4 mg/ml. Among these embodiments are unit dosage forms at concentrations of 3 mg/ml or 4 mg/ml in volumes of 1 ml or of 2 ml, providing unit dosage forms comprising 3 mg dipyridamole in 1 ml, 6 mg dipyridamole in 2 ml, 4 mg in 1 ml and 8 mg dipyridamole in 2 ml.

In some embodiments, the dipyridamole concentration is 1 mg/ml or 2 mg/ml.

In some embodiments the dipyridamole unit dosage forms are prefilled syringes, optionally with kilo graduation marks on their cylinder to facilitate weight-adjusted dosing. The scale of such a graduation marks, provided in some embodiments by an affixed label, can range, for example, from 10-40 to 100 or 200 kilograms with an interval of 1 kilogram=0.01 ml whatever the features of the syringe used.

In one aspect, unit dosage forms of adenosine are provided. In some embodiments, unit dosage forms are formulated in sterile fluid composition, and dose packaging permits sterile introduction of a second fluid in a volume at least 15% that of the adenosine composition. In some embodiments, the second fluid usefully comprises dipyridamole (see, e.g., Table 2 and FIG. 5).

In exemplary embodiments, the unit dosage form contains 21 mg adenosine in 6 ml; 28 mg adenosine in 6 ml; 42 mg adenosine in 12 ml; or 56 mg adenosine in 12 ml (see Table 2).

In certain embodiments, the composition in the unit dosage form has a pH less than about 4.5, less than about 4.4, less than about 4.3, less than about 4.2, less than about 4.1, less than about 4.0, less than about 3.9, less than about 3.8, less than about 3.7, even less than about 3.6, including intermediate nonintegral pH values therebetween. In certain embodiments, the pH is between about 2 and 4.

In certain exemplary embodiments of the methods presented herein, unit dosage forms of adenosine are administered immediately after a dipyridamole bolus injection. In some embodiments unit dosage forms (vials or prefilled syringes) contain 14 mg adenosine in 10 ml, 21 mg adenosine in 10 ml, 28 mg adenosine in 10 ml, 35 mg in 15 ml, 42 mg in 20 ml, 56 mg in 20 ml. In some embodiments adenosine is at a concentration of about 3 mg/ml, with 21 mg adenosine in 7 ml or 42 mg adenosine in 14 ml, or the unit dosage form may comprise adenosine at 4 mg/ml with 28 mg adenosine in 7 ml or 56 mg adenosine in 14 ml.

In some embodiments, when the unit dosage forms are in the form of prefilled syringes, the barrel of these syringes can usefully be labeled with weight graduation marks to permit convenient weight-adjusted dosing. In some embodiments, the weight graduation scale is in kilograms. These kilo graduation scales can go from 10 or 40 kilograms up to 100 or 125 or 133 or 150 or 166 or even 200 kilograms depending on the syringe capacity. Milliliter per kilogram equivalence can range from 1 kilogram=0.0005 ml, to 1 kilogram=1 ml regardless of the syringe characteristics and more typically from 1 kilogram=0.01 ml, to 1 kilogram=0.2 ml. In some embodiments, the milliliter per kg equivalence is of 0.06 ml or 0.075 ml or 0.08 ml or 0.09 ml or 0.1 ml or 0.12 ml, regardless of the syringe characteristics.

In one embodiment, adenosine at a concentration of 3 mg/ml is provided in a prefilled syringe with weight graduation marks that contains 30 mg adenosine in a volume of 10 ml. In one embodiment, a prefilled syringe comprises a label with a kilo graduation scale where a 1 kg interval is equivalent to 0.07 ml if infusion time is 3 minutes, the 7 ml marking equals 100 kg (or the dose for a patient weighing 100 kg) and the 10 ml marking equals 130 kilograms (129.870 kgs).

In some embodiments, the unit dosage form comprises a pre-filled syringe comprising 60 mg adenosine in a volume of 20 ml. In some embodiments, the unit dosage form comprises a pre-filled syringe comprising 90 mg adenosine in a volume of 30 ml. The pre-filled syringe, in various embodiments, further comprises labels as described herein.

In some embodiments, unit dosage forms of adenosine are administered in parallel with dipyridamole through separate interdependent (yoked) syringes (see, e.g., FIG. 6). In these embodiments, the unit dosage form of adenosine and the unit dosage form of dipyridamole may each comprise any of the prefilled syringes described herein.

In certain of these embodiments a specific connector is used so that a fraction of the dipyridamole dose flows into the venous line prior to adenosine, thereby serving as priming dose. The connector also serves as a mixer when, immediately after this priming dose, the concurrent infusion of the two drugs becomes effective.

Also provided are kits. The kits comprise at least one unit dosage form of dipyridamole and at least one unit dosage form of adenosine. The unit dosage forms can be prefilled syringes. In some embodiments, the at least one unit dosage form of dipyridamole is a unit dosage form as above-described, and the unit dosage form of adenosine is a unit dosage form as above-described. Adapted connectors and extension set/venous lines are usefully included in the kits.

In some kit embodiments the kit comprises one unit dosage form of the adenosine:dipyridamole composition. In other embodiments the kits comprise at least one unit dosage form of adenosine and at least one unit dosage form of dipyridamole. The unit dosage forms can be prefilled syringes. In some embodiments, the at least one unit dosage form of adenosine:dipyridamole composition is a unit dosage form as above-described, and the unit dosage forms of dipyridamole and of adenosine are unit dosage forms as above-described. Adapted connectors, diluent (e.g. saline) and extension set/venous lines are usefully included in the kits.

In summary, adenosine (Adenoscan, sold by Astellas) is the standard pharmacological stressor used in cardiac imaging to induce near-maximal coronary vasodilation. At its recommended dosage rate of 140 μg/kg/min, its use is attended by numerous uncomfortable side effects. The data from the studies presented herein demonstrate that the sequential bolus administration of dipyridamole at 28-40 μg/kg—well below the total dose infused when dipyridamole is used as a single agent stressor—followed by infusion of adenosine at 70 μg/kg/min, is equally efficacious in providing coronary vasodilation for imaging studies, while causing fewer side effects. The data also demonstrate that dipyridamole and adenosine may be combined in a single infusion (in particular 10 μg/kg/min dipyridamole with 70 μg/kg/min adenosine), over 4 minutes and possibly over a 3 minute or even 2 minute period, to similar effect. Among the side effects reduced by the combination of the present invention are chest pain and the risk of significant heart blockage.

### 3. BRIEF DESCRIPTION OF THE DRAWINGS

The above and other objects and advantages of the present invention will be apparent upon consideration of the following detailed description taken in conjunction with the accompanying drawings, in which like characters refer to like parts throughout, and in which:

FIGS. 1A-1E present exemplary embodiments of labels for weight-adjusted dosing of pharmaceutical compositions according to embodiments of the present invention. FIG. 1A shows an exemplary syringe with label for weight-adjusted dosing. The barrel of the syringe unit (010) contains a scale (020) having graduation marks on one or both sides. A double scale is shown having marks for measurements in two different units, volume and weight. Any known unit of weight can be used for the weight graduation scale on a label, including but not limited to, kilograms (kg) or pounds (lbs). The inset shows an expanded view of a double scale with graduation marks for two measurements, volume and weight. FIG. 1B illustrates that the weight scale in various embodiments can range as low as 10 kilograms, approximately 22 pounds, or an equivalent weight in any other known unit of measurement. FIG. 1C illustrates that weight graduations can range up to any desired maximum, for example, weights of 150 and 200 kg, respectively. Equivalent weight graduation scales are possible in other units of measurement, including pounds (such as 330 or 440 lbs, respectively, for the example provided). FIG. 1D provides an example of dual volume and weight scale, with offset graduations. In some embodiments, the label comprises both a volume and a weight scale, which are offset relative to each other. FIG. 1E illustrates an example of a label having a single scale with units of weight, in kilograms. Labels comprise at least a weight scale for dosing. In some cases, the scale is provided in units of pounds;

FIGS. 2A and 2B illustrate exemplary syringe labels with kilogram-graduation scales for use in sequential administration of pharmaceutical compositions according to embodiments of the present invention. FIG. 2A illustrates an exemplary labeled syringe useful in initial administration of a bolus of dipyridamole (4 mg in 1 ml unit dosage form, “UDF”), dose 40 mg/kg, according to various embodiments of the methods of the present invention. Two different examples of labels are provided, one with a 40 to 100 kg scale, and the second with a 10 to 100 kg scale. In each case, the scale is calibrated such that 0.01 ml is administered for every kilogram of patient weight. FIG. 2B illustrates an exemplary labeled syringe useful in adenosine infusion to be made subsequent to the dipyridamole administration, according to various embodiments of the methods of the present invention. In these examples, adenosine (28 mg per 10 ml UDF) is infused, dose 70 mg/kg/min. Two different examples are shown, one for a 4-minute infusion and the second for a 3-minute infusion. In the 4-minute infusion, the scale is calibrated such that 0.1 ml is administered for every kilogram of patient weight. For a 3-minute infusion, the scale is calibrated such that 0.075 ml is administered for every kilogram of patient weight;

FIGS. 3A and 3B illustrate an example of sequential administration, according to various embodiments of the present invention. In FIG. 3A., dipyridamole is injected intravenously, e.g. as a 5- to 30-second bolus (mean injection time 5-10 seconds), via Y-connector leading to an intravenous line. In FIG. 3B, adenosine is infused immediately subsequent to dipyridamole administration using a programmable delivery device, such as an electric pump. Typical infusion times include infusions of 3 and 4 minutes;

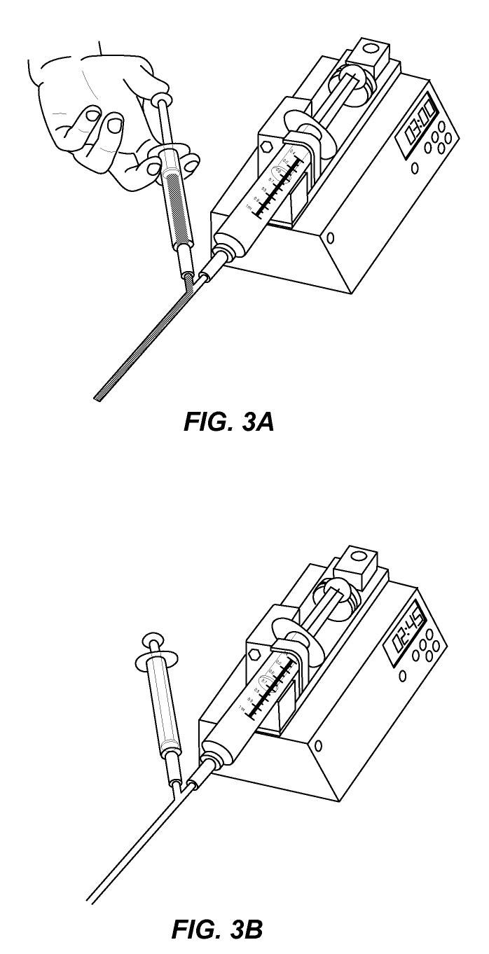

FIGS. 4A-4C illustrate the exemplary adjustment and individualization, prior to use, of an exemplary prefilled syringe unit dosage containing a combined composition of adenosine and dipyridamole. In FIG. 4A, dose is first adjusted based on patient weight, given the amount of each of the actives in the prefilled syringe. In the exemplary embodiment shown, a 10-ml syringe is provided prefilled with 28 mg adenosine and 4 mg dipyridamole in a 4 ml volume. In FIG. 4B, saline is added, e.g. to adjust pH in embodiments in which the combined composition has an acidic pH. In certain embodiments, the optimum ratio of saline solution to composition comprising the actives is about 1:2. As shown in the example, at least 6 ml of saline is added to adjust the volume to a total of 10 ml. FIG. 4C illustrates the connection to an extension set of the syringe containing the appropriate, pH-adjusted and weight-adjusted dose in a convenient volume, ready for infusion according to methods of the present invention;

FIGS. 5A-5D illustrate exemplary embodiments in which dipyridamole and adenosine are administered concurrently according to embodiments of the present invention, using a prefilled unit dose syringe of adenosine pharmaceutical composition and a unit dose of dipyridamole pharmaceutical composition separately packaged in a vial. In FIG. 5A, dipyridamole (4 mg in 1 ml) UDF is added to a prefilled 10-ml syringe containing 28 mg adenosine in 6 ml volume. FIG. 5B shows an optional addition of saline to a final volume of 10 ml to facilitate administration via programmable delivery device, such as an electric pump. In FIG. 5C, the dose is adjusted based on the weight of the patient, using an exemplary weight graduation scale, e.g., as imprinted on a syringe label, as exemplified in FIGS. 1 and 2. In FIG. 5D, the dosage form so prepared is connected to extension set for infusion; and

FIGS. 6A and 6B illustrate two different exemplary syringe system embodiments (respectively Model A and Model B) for concurrent infusion of adenosine and dipyridamole according to embodiments of the present invention. Models A and B depict examples of interdependent syringes not in fluid communication with one another, optionally provided with labels for weight-adjusted dosing on the adenosine-containing syringe.

### 4. DETAILED DESCRIPTION

### 4.1. Overview

Although dipyridamole is believed to act indirectly by increasing endogenous adenosine concentration, and in clinical practice has a side effect profile similar to that of adenosine, and although dipyridamole is also known to potentiate exogenous adenosine adverse events to such an extent that its combination with adjunctively administered adenosine is normally contraindicated (“drugs that augment the effects of adenosine should be withheld for at least five half-lives prior to the use of Adenoscan”, quoted from Adenoscan drug label), I have now discovered that extremely low parenteral-subclinical doses of dipyridamole—on the order of 5% of the dose now used clinically in cardiac imaging studies—can potentiate the vasodilation effects of adjunctively administered adenosine without commensurate potentiation of adenosine's side effects. This permits adenosine to be used at reduced dosage to effect coronary vasodilation, e.g., for functional assessment of myocardial function, with equal or superior efficacy as compared to current protocols, yet with reduced side effects, of short duration.

Adenosinergic agents other than, or additional to, adenosine—herein collectively termed “adenosine receptor agonists”—can be used. Such agonists are usefully selected from the group consisting of adenosine, and adenosine donors (that is, compounds that can be metabolized to adenosine), including natural donors such as adenosine triphosphate (ATP), adenosine diphosphate (ADP), and adenosine monophosphate (AMP), each at approximately the same dosages as adenosine, and any synthetic molecule that is capable of being metabolized to adenosine, and pharmaceutically acceptable salts thereof. For convenience of reference, and not by way of limitation, adenosine will typically be described as the adenosine receptor agonist for use in the methods, compositions, and kits herein.

Agents other than, or additional to, dipyridamole, can likewise be adjunctively administered at sub-maximal dosage to potentiate the desired coronary vasodilatory effects of the adenosine receptor agonist with reduced side effects. Such dipyridamole-like agents, herein collectively termed “adenosine modulators”, interfere with plasma clearance of adenosine, either by inhibiting or slowing adenosine uptake by red blood cells and/or endothelial cells, or by inhibiting enzymes involved in adenosine anabolic or catabolic metabolism.

For purposes of myocardial perfusion imaging studies, which are of short duration, preferred adenosine modulators act to reduce or inhibit the capture of adenosine by erythrocytes and/or endothelial cells. The modulators may act, e.g., through competition with, or noncompetitive inhibition of, cell-membrane transporters.

Adenosine modulators useful in the present invention include, in addition to dipyridamole, hexobendine, dilazep, lidoflazine, draflazine, nitrobenzylthioinosine (NBMPR), nitrobenzylthioguanosine, p-nitronezylthioguanosine (NBTGR), and adenosine analogues such as adenosine-5′-carboxamides and xylosyladenosine, and pharmaceutically acceptable salts and derivatives thereof. Other adenosine modulators useful in the present methods include cilostazol, a quinolone derivative, acadesine and papaverine, and pharmaceutically acceptable salts and derivatives thereof. These latter agents, in addition to their established phosphodiesterase inhibitory activity, are also thought to be nucleoside transporter inhibitors. Some of the adenosine modulators listed above are currently available clinically, in the U.S. and/or Europe, such as cilostazol, papaverine, dilazep, and draflazine, generally in the form of pills for oral administration, some also formulated for injection (e.g., papaverine). Other adenosine modulators inhibit metabolic enzymes, such as adenosine kinase (which converts adenosine into AMP) or adenosine deaminase (which converts adenosine into inosine).

For convenience of reference, and not by way of limitation, dipyridamole will typically be described as the adenosine modulator for use in the methods, compositions, and kits herein. When dipyridamole is the adenosine modulator and adenosine is the adenosine receptor agonist, the combination—at any dose ratio—will be referred to herein as Adenosoft.

Example 1, hereinbelow, compares the hemodynamic effects of administering dipyridamole and adenosine intravenously as a combined (sequentially administered) pharmacological stressor, to the effects of administering adenosine alone in 40 consecutive patients suffering from ischemic heart disease. Each patient served as his own control. Dipyridamole was administered as an intravenous bolus. Adenosine was administered immediately thereafter by continuous intravenous infusion for three minutes. Each of the two agents was administered at a dosage lower than its clinically preferred dosage when used as a single agent for myocardial perfusion imaging: dipyridamole at 4-6% of its single-agent total dose, adenosine at one half its single-agent dosage rate.

Effects were measured using noninvasive transthoracic doppler echocardiography (TTDE). The measured blood flow velocities (known to reflect coronary blood flow values), whether peak or mean, were 1.5 to 4% lower in absolute values than those measured upon administration of adenosine alone at its standard dosage rate. However, these differences were not statistically significant (p>0.05): there was no statistical difference between the current standard treatment—infusion of adenosine alone at 140 μg/kg/min—and sequential bolus administration of dipyridamole at 4-6% of its typical single-agent total dose followed by adenosine infusion at 70 μg/kg/min.

In addition to this series of 40 patients, three (3) patients (excluded from the statistical analysis) received the adenosine infusion two minutes after the dipyridamole bolus, rather than immediately thereafter, and two (2) patients (also excluded from the statistical analysis) were injected with the two agents concurrently in the same infusion line using a Y-shaped, or Y-, connector. No differences were seen as compared to the sequential administration protocol.

A significant reduction was seen in the incidence of chest pain among the 40 patients in this study, as compared to the number reporting chest pain upon administration of adenosine alone at 140 μg/kg/min. In addition, the severity of the three main adverse side effects—chest pain, dyspnea, and flushing—cumulated across all dipyridamole doses, was reduced by 31.6% with the sequential combination as compared to standard adenosine treatment. This decrease was statistically significant (p=0.001).

As reported in detail in Example 2, 56 patients were assessed in a subsequent Phase II study comparing in a single blind, 2-arm cross over protocol design dipyridamole-adenosine combination administration to adenosine alone (Adenoscan®, Astellas) as the pharmacologic stressor in coronary patients undergoing single photon emission computed tomography (SPECT) imaging studies.

Study results showed that either the sequential or concomitant IV administration (premixing) of dipyridamole microdoses at 5% the standard dose, with adenosine (Adenoscan) at 50% its maximal dose, during 4 minutes were statistically equivalent in terms of efficacy (severity score comparison, agreement rate for the presence of perfusion defects and categorical agreement rate) to the reference drug infused during 6 minutes: in endpoint terms the combination is noninferior to Adenoscan (p<0.0001).

Significant reduction in both the occurrence and the severity of chest pain with the dipyridamole-adenosine combination, as compared to adenosine alone, was observed, as was reduction in ST changes on EKG. The occurrence of chest pain (A1 side effect) was significantly reduced by 43% (p=0.03) and severity by 61% (p=0.001). The occurrence of A2 side effects with Adenosoft was clearly reduced: by 23% for dyspnea and 24% for flushing. The severity of dyspnea and flushing was reduced by 49% and 51% respectively (p=0.01 and p=0.03). The Adenosoft composite index (cumulative severity of chest pain, dyspnea and flushing) was significantly below that of Adenoscan (−53%, p<0.0001).

The data from these first two studies demonstrate (i) that the sequential bolus administration of dipyridamole at 28 to 40 μg/kg—acting as a pretreatment and well below the total dose infused when dipyridamole is used as a single agent stressor—followed by infusion of adenosine at 70 μg/kg/min (50% less than its usual dosage) over 3 minutes, is equally efficacious in providing coronary vasodilation for imaging studies, while causing fewer side effects. The data also demonstrate that dipyridamole and adenosine may be combined in a single infusion, over 4 minutes, to similar effect. Among the side effects reduced by the combination of the present invention are chest pain, and the risk of significant heart blockage.

Two additional studies were conducted in 43 coronary patients assessed for coronary reserve using transthoracic-doppler-echography. These studies demonstrated that the concurrent administration of adenosine at 70 μg/kg/min with dipyridamole at 10 μg/kg/min during a period of only two minutes is statistically equivalent, with respect to efficacy, to adenosine infused at its recommended dose of 140 μg/kg/min. Side effects, especially A2a adverse events (dyspnea and flushing), appeared to be further reduced with the 2 minute infusion as compared to the longer infusion times used in the first two studies. Time to peak to achieve near maximal hyperemia occurs after 1 minute or so of infusion with adenosine alone (Adenoscan) and after about 1.5 minute with the combination. Intra-individual measurements showed that in the same patient return to base line with the combination occurs 10 to 15 seconds later than with adenosine infusion alone. However, time to peak is on average close to 1 minute and similar to that of adenosine alone (with unchanged return to baseline), when a fraction of the total dipyridamole dose, acting as a priming dose, in the 0.1-0.3 mg range, is administered prior to the combination infusion. The same is true when the sequential administration mode is used over 2 minutes as was also demonstrated in a series of 10 consecutive patients (see example 5.5).

These latter data demonstrated that dipyridamole and adenosine may be combined as a single 2 minute infusion with potentially further reduction of side effects and maintained efficacy. When infusion time is shortened by one minute and possibly more, efficacy, which very much depends on time to peak, can be usefully secured by either: i) administering a priming dose of dipyridamole (actually a fraction of dipyridamole total dose) administered as pretreatment prior (i.e., less than a few seconds and sometimes less than one second) to the adenosine-dipyridamole combination infusion; or ii) by the sequential administration mode with dipyridamole (e.g., 40 μg/kg) administered first as a 5-20 seconds bolus rapidly followed by adenosine infusion at 70 μg/kg/min over 2 to 3 minutes.

Accordingly, described herein are methods, pharmaceutical compositions, unit dosage forms, and kits that exploit this discovery, combining adenosine with dipyridamole (or, in various embodiments, other adenosine modulator) at dosages at which some of the most frequent side effects of both adenosine and dipyridamole, notably cardiac side effects, are significantly reduced, while maintaining optimal coronary vasodilation for the diagnosis of myocardial ischemia.

### 4.2. Methods of Effecting Coronary Vasodilation

In one aspect, methods of effecting coronary vasodilation for cardiac diagnosis are provided.

In typical embodiments, the methods comprise (i) parenterally administering an adenosine modulator, such as dipyridamole, and sequentially thereafter parenterally administering an adenosine receptor agonist, such as adenosine, or (ii) concurrently administering an adenosine modulator, such as dipyridamole, and an adenosine receptor agonist, such as adenosine, optionally with a fraction of the total modulator dose administered prior to this concurrent administration. Each of the adenosine modulator, e.g., dipyridamole, and the adenosine receptor agonist, e.g., adenosine, is administered at a dosage lower than that required for maximal coronary vasodilation when the respective agent is administered individually by identical parenteral route. The adenosine modulator and the adenosine receptor agonist are administered in amounts, at weight ratios, and for a time, sufficient to achieve the desired therapeutic or diagnostic effect.

Programmable syringe pumps or micropumps, as are typical in clinical practice, are usefully employed to facilitate parenteral administration in precise dosage. However manual administration is also possible when infusion time does not exceed 3 minutes.

The route of parenteral administration is chosen based upon the desired clinical effect, as further described below. In certain embodiments, at least one of the adenosine modulator, e.g., dipyridamole, and the adenosine receptor agonist, e.g., adenosine, is administered by intravenous infusion. In most embodiments the two active agents are administered intravenously. In other embodiments, at least one of dipyridamole and the adenosine receptor agonist is administered by intra-arterial infusion, such as intra-coronary infusion, or by intra-atrial infusion. In these latter embodiments, the active is administered at a lower rate, and at a lower dosage, than for intravenous infusion, as further described below. In yet other embodiments, at least one of the actives is administered as a perfusate.

In some embodiments, at least one of dipyridamole and the adenosine receptor agonist is infused over a period of time of at least 1 minute, typically at least 2 minutes, 3 minutes, 4 minutes, 5 minutes, even at least 6 minutes. As used herein, “continuous infusion” intends infusion over a period of at least 2 minutes.

In some embodiments, dipyridamole is administered by intravenous infusion at an infusion rate from 3.5 μg/kg/min to 50 μg/kg/min. All dosage ranges described herein include the upper and lower recited limits, and nonintegral intermediary values. Thus, in some embodiments, dipyridamole is infused at a rate of at least about 3.5 μg/kg/min, at least about 4 μg/kg/min, at least about 5 μg/kg/min, at least about 6 μg/kg/min, at least about 7 μg/kg/min, at least about 7.5 μg/kg/min, at least about 8 μg/kg/min, at least about 8.75 μg/kg/min, at least about 9 μg/kg/min, at least about 10 μg/kg/min, at least about 11 μg/kg/min, at least about 11.25 μg/kg/min, at least about 12 μg/kg/min, at least about 12.5 μg/kg/min, at least about 13 μg/kg/min, at least about 13.75 μg/kg/min, at least about 14 μg/kg/min, at least about 15 μg/kg/min, at least about 16 μg/kg/min, at least about 16.25 μg/kg/min, at least about 17 μg/kg/min, and at least about 17.5 μg/kg/min, at least about 18 μg/kg/min, at least about 19 μg/kg/min, at least about 20 μg/kg/min, at least about 21 μg/kg/min, at least about 22 μg/kg/min, at least about 23 μg/kg/min, at least about 24 μg/kg/min, at least about 25 μg/kg/min, at least about 26 μg/kg/min, at least about 27 μg/kg/min, at least about 28 μg/kg/min, at least about 29 μg/kg/min, at least about 30 μg/kg/min, at least about 31 μg/kg/min, at least about 32 μg/kg/min, at least about 33 μg/kg/min, at least about 34 μg/kg/min, at least about 35 μg/kg/min, at least about 36 μg/kg/min, at least about 37 μg/kg/min, at least about 38 μg/kg/min, at least about 39 μg/kg/min, at least about 40 μg/kg/min, at least about 41 μg/kg/min, at least about 42 μg/kg/min, at least about 43 μg/kg/min, at least about 44 μg/kg/min, at least about 45 μg/kg/min, at least about 46 μg/kg/min, at least about 47 μg/kg/min, at least about 48 μg/kg/min, at least about 49 μg/kg/min, at least about 50 μg/kg/min, with intermediate values permissible.

In some embodiments, dipyridamole is infused intravenously at a rate of no more than about 50 μg/kg/min, no more than about 49 μg/kg/min, no more than about 48 μg/kg/min, no more than about 47 μg/kg/min, no more than 46 μg/kg/min, no more than about 45 μg/kg/min, no more than about 44 μg/kg/min, no more than about 43 μg/kg/min, no more than about 42 μg/kg/min, no more than about 41 μg/kg/min, no more than about 40 μg/kg/min, no more than about 39 μg/kg/min, no more than about 38 μg/kg/min, no more than about 37 μg/kg/min, no more than about 36 μg/kg/min, no more than about 35 μg/kg/min, no more than about 34 μg/kg/min, no more than about 33 μg/kg/min, no more than about 32 μg/kg/min, no more than about 31 μg/kg/min, no more than about 30 μg/kg/min, of no more than about 29 μg/kg/min, no more than about 28 μg/kg/min, no more than about 27 μg/kg/min, no more than about 26 μg/kg/min, no more than about 25 μg/kg/min, no more than about 24 μg/kg/min, no more than about 23 μg/kg/min, no more than about 22 μg/kg/min, no more than about 21 μg/kg/min, no more than about 20 μg/kg/min, no more than about 19 μg/kg/min, no more than about 18 μg/kg/min, no more than about 17.5 μg/kg/min, no more than about 17 μg/kg/min, no more than about 16.25 μg/kg/min, no more than about 16 μg/kg/min, no more than about 15 μg/kg/min, no more than about 14 μg/kg/min, no more than about 13.75 μg/kg/min, no more than about 13 μg/kg/min, no more than about 12.5 μg/kg/min, no more than about 12 μg/kg/min, no more than about 11.25 μg/kg/min, no more than about 11 μg/kg/min, no more than about 10 μg/kg/min, no more than about 9 μg/kg/min, no more than about 8.75 μg/kg/min, no more than about 8 μg/kg/min, no more than about 7.5 μg/kg/min, no more than about 7 μg/kg/min, no more than about 6 μg/kg/min, no more than about 5 μg/kg/min, no more than about 4 μg/kg/min, no more than about 3.5 μg/kg/min, with intermediate values permissible.

In some embodiments, dipyridamole is administered as a bolus, typically over a period of about 5-30 seconds.

In some of these embodiments, dipyridamole is administered as an intravenous bolus. In such embodiments, dipyridamole is administered at a dosage between 14 μg/kg to 140 μg/kg. In various embodiments, dipyridamole is administered intravenously as a bolus at a dosage between 28 μg/kg and 40 μg/kg, 40 μg/kg being the preferred dosage.

Thus, in certain embodiments, dipyridamole is administered as an intravenous bolus at a dose of at least about 14 μg/kg, at least about 20 μg/kg, at least about 25 μg/kg, at least about 28 μg/kg, at least about 29 μg/kg, at least about 30 μg/kg, at least about 31 μg/kg, at least about 32 μg/kg, at least about 33 μg/kg, at least about 34 μg/kg, at least about 35 μg/kg, at least about 36 μg/kg, at least about 37 μg/kg, at least about 38 μg/kg, at least about 39 μg/kg, at least about 40 μg/kg, at least about 45 μg/kg, at least about 50 μg/kg, at least about 55 μg/kg, at least about 60 μg/kg, at least about 65 μg/kg, even at least about 70, 80, 90, 100, 110, 120, 130, even 140 μg/kg, with intermediate doses permissible.

In some embodiments, dipyridamole is administered intravenously as a bolus at a dosage of no more than about 140 μg/kg, 130 μg/kg, 120 μg/kg, 110 μg/kg, 100 μg/kg, 90 μg/kg, 80 μg/kg, 70 μg/kg, even no more than about 60 μg/kg, even no more than about 55 μg/kg, no more than about 50 μg/kg, no more than about 45 μg/kg, no more than about 40 μg/kg, no more than about 39 μg/kg, no more than about 38 μg/kg, no more than about 37 μg/kg, no more than about 36 μg/kg, no more than about 35 μg/kg, no more than about 34 μg/kg, no more than about 33 μg/kg, no more than about 32 μg/kg, no more than about 31 μg/kg, no more than about 30 μg/kg, no more than about 29 μg/kg, no more than about 28 μg/kg, no more than about 25 μg/kg/, no more than about 20 μg/kg/, no more than about 14 μg/kg, with intermediate values permissible.

When administered to a human being, the dosages of dipyridamole useful in the methods of the present invention can be expressed in μg by multiplying the dosage, expressed as μg/kg, by the weight of the individual. For example, for a human weighing 50 kg, the dosage of dipyridamole useful in the present methods can be expressed as ranging between 700 to 7,000 μg; for a human being weighing 60 kg, the dosage of dipyridamole can be expressed as ranging between 840 to 8,400 μg; for a human being weighing 75 kg, the dosage of dipyridamole can be expressed as ranging between 1,050 to 10, 500 μg; and for a human being weighing 100 kg, the dosage can be expressed as ranging between 1,400 to 14,000 μg.

In various embodiments, dipyridamole is infused intra-arterially at an infusion rate of no more than about 0.07 μg/kg/min, no more than about 0.06 μg/kg/min, no more than about 0.05 μg/kg/min, no more than about 0.04 μg/kg/min, no more than about 0.03 μg/kg/min, no more than about 0.02 μg/kg/min, or no more than about 0.01 μg/kg/min, with intermediate values permissible.

In various embodiments, the adenosine receptor agonist is selected from the group consisting of adenosine, and adenosine donors (that is, compounds that can be metabolized to adenosine), including natural donors such as adenosine triphosphate (ATP), adenosine diphosphate (ADP), and adenosine monophosphate (AMP), each at approximately the same dosages as adenosine, and any synthetic molecule that is capable of being metabolized to adenosine, and pharmaceutically acceptable salts thereof.

Typically, adenosine is used. For convenience, its particular use will hereafter be described, without intending thereby to limit the described methods to use of adenosine as the adenosine receptor agonist.

In some embodiments, adenosine is administered by intravenous infusion at an infusion rate between 35 μg/kg/min to 100 μg/kg/min. Thus, in some embodiments, adenosine is infused at a rate of at least about 35 μg/kg/min, at least about 40 μg/kg/min, at least about 45 μg/kg/min, at least about 50 μg/kg/min, at least about 55 μg/kg/min, at least about 60 μg/kg/min, at least about 65 μg/kg/min, at least about 70 μg/kg/min, at least about 75 μg/kg/min, at least about 80 μg/kg/min, at least about 85 μg/kg/min, at least about 90 μg/kg/min, at least about 95 μg/kg/min, and at least about 100 μg/kg/min, with intermediate values permissible.

In various embodiments, adenosine is infused intravenously at a rate of no more than about 100 μg/kg/min, no more than about 95 μg/kg/min, no more than about 90 μg/kg/min, no more than about 85 μg/kg/min, no more than about 80 μg/kg/min, no more than about 75 μg/kg/min, no more than about 70 μg/kg/min, no more than about 65 μg/kg/min, no more than about 60 μg/kg/min, no more than about 55 μg/kg/min, no more than about 50 μg/kg/min, no more than about 45 μg/kg/min, no more than about 40 μg/kg/min, no more than about 35 μg/kg/min, with intermediate values permissible.

When administered to a human being, the dosage rate of adenosine can be expressed in μg/min by multiplying the dosage rate expressed in μg/kg/min by the weight of the individual. For example, for a human being weighing 50 kg, the dosage of adenosine useful in the practice of the present methods can be expressed as ranging between 1,750 to 5,000 μg/min; for a human being weighing 60 kg, the dosage rate of adenosine can be expressed as ranging between 2,100 to 6,000 μg/min; for a human being weighing 75 kg, the dosage of adenosine can be expressed as ranging between 2,625 to 7,500 μg/min; and for a human being weighing 100 kg, the dosage of adenosine can be expressed as ranging between 3,500 to 10,000 μg/min.

In some embodiments, adenosine is administered by intra-arterial infusion, such as intracoronary infusion, at an infusion rate about 200- to 400-fold lower than intravenous infusion. Thus, in some embodiments, adenosine is infused at a rate of at least about 0.50 μg/kg/min, at least about 0.45 μg/kg/min, at least about 0.40 μg/kg/min, 0.35 μg/kg/min, at least about 0.30 μg/kg/min, at least about 0.25 μg/kg/min at least about 0.20 μg/kg/min, at least about 0.15 μg/kg/min, at least about 0.10 μg/kg/min, with intermediate values permissible.

In various embodiments, adenosine is infused intra-arterially and in particular by intracoronary infusion, at an infusion rate of no more than about 0.10 μg/kg/min, no more than about 0.15 μg/kg/min, no more than about 0.20 μg/kg/min, no more than about 0.25 μg/kg/min, no more than about 0.30 μg/kg/min, no more than about 0.35 μg/kg/min, no more than about 0.40 μg/kg/min, no more than about 0.45 μg/kg/min, even no more than about 0.50 μg/kg/min, with intermediate values permissible.

In various embodiments, the methods presented herein comprise parenterally administering dipyridamole and sequentially thereafter parenterally administering an adenosine receptor agonist, such as adenosine at an adenosine:dipyridamole (A:D) weight ratio of about 2:1 to about 10:1. In other embodiments, the methods comprise concurrently administering adenosine and dipyridamole, without or with a dipyridamole priming dose at adenosine:dipyridamole weight ratio of about 2:1 to about 10:1.

In some embodiments, the, ratio is about 2:1,3:1,4:1,5:1,6:1,7:1,8:1,9:1, even 10:1, with nonintegral ratios between about 2:1 and about 10:1 permissible. In certain embodiments, the methods comprise concurrent infusion of adenosine and dipyridamole, with or without pre-treatment by a priming dose of dipyridamole or the sequential administration of the two drugs, at an A:D ratio of about 6:1 to 8:1, preferably about 7:1. For certain methods further described below, embodiments usefully comprise concurrent or sequential parenteral infusion of adenosine and dipyridamole at an A:D weight ratio of about 2:1 to 4:1.

In various embodiments the sequential method is used: dipyridamole is administered first as an intravenous bolus, and adenosine is administered thereafter as an intravenous infusion. In some embodiments, adenosine is administered into a coronary artery at a dose of about 10 to about 20 μg/min, regardless of patient weight, after intravenous bolus injection of dipyridamole at a dose of about 20 to about 40 μg/kg, preferably 40 μg/kg.

In certain such embodiments, dipyridamole is administered over about 5-30 seconds, and adenosine is thereafter infused for about 1 to 6 minutes.

In these embodiments, the total dose of dipyridamole given intravenously as a bolus is typically between about 1/16 to about 1/24, e.g., about 1/20 (5%), that of the total recommended standard dose when dipyridamole is used as a single agent (standard single agent dose: 0.56 mg-0.80 mg/kg). In these embodiments, the total dose of intravenously infused adenosine is typically about 25% to about 50% that of the total recommended standard dose when adenosine is used as a single agent (standard single agent dose: 0.84 mg/kg).

In certain embodiments, dipyridamole is administered as an IV bolus over about 5-30 seconds at a dosage of about 14 to 60 μg/kg, followed immediately (that is, as soon as clinically practicable, typically within about 5 to 30 seconds) by the infusion of adenosine at a dosage of about 35 to 100 μg/kg/min for a period of about 1 to 6 minutes. The duration of adenosine administration is determined by the chosen imaging methodology, as is well known in the art.

In some embodiments, an intravenous dipyridamole bolus of 28-40 μg/kg is followed immediately—that is, as soon as clinically practicable, typically within about 5 to 30 seconds—by intravenous infusion of adenosine at 50-70 μg/kg/min for 1 to 4 minutes. In certain embodiments, an IV dipyridamole bolus of 40 μg/kg is followed immediately by intravenous administration of adenosine at 70 μg/kg/min for 3 or 4 minutes. In some embodiments, the adenosine administration is for a period of about 1 or 2 minutes.

In sequential administration embodiments, adenosine infusion may be delayed as long as about 2-10 minutes after dipyridamole bolus, typically no more than about 5 minutes after dipyridamole bolus.

In sequential administration embodiments, dipyridamole may be injected manually as a bolus via a syringe, or automated by the use of a programmable device (e.g., by micropump). When administered by micropump, dipyridamole may be injected over about 1 to about 2 minutes prior to adenosine infusion. Adenosine infusion is typically accomplished using a programmable device so as to ensure its measured delivery. However, manual administration over about 2 to about 3 minutes may also be preformed.

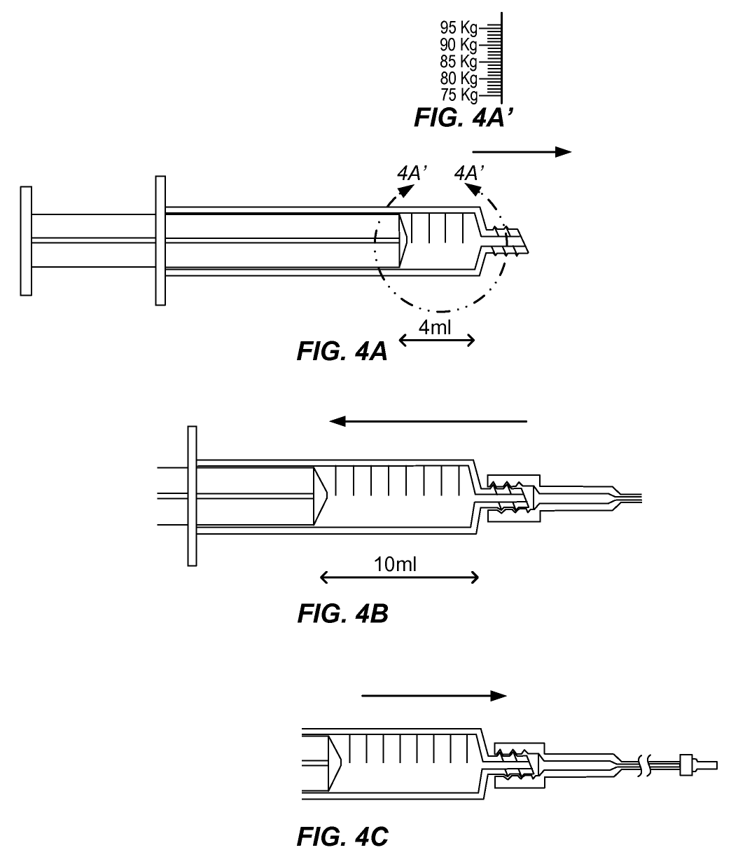

In some embodiments, dipyridamole and adenosine are administered concurrently (see, e.g., FIGS. 4 to 6) over about 1 to about 6 minutes. The duration of intravenous administration is determined by the chosen imaging methodology, as is well known in the art. In some embodiments the duration can be of either about 3 or about 4 minutes. In other embodiments of either about 1 or about 2 minutes.

In certain administration embodiments, dipyridamole and adenosine are combined from separate unit dosage forms and then administered concurrently as a single composition.

For example, in some embodiments, a volume of dipyridamole corresponding to a dosage of 14 to 60 μg/kg is sampled and a volume of adenosine corresponding to a dosage of 35 to 100 μg/kg/min is similarly sampled and the two mixed in the same syringe. In some embodiments, a volume of dipyridamole corresponding to 28-40 μg/kg is sampled and a volume of adenosine corresponding to a dosage of 50 to 70 μg/kg/min is similarly sampled, and the two mixed in the same syringe. In certain embodiments, a volume of dipyridamole corresponding to 40 μg/kg is sampled and a volume of adenosine corresponding to a dosage of 70 μg/kg/min is similarly sampled and the two mixed in the same syringe. Thus, as is described further below, in another aspect, specific unit dosage forms of adenosine are provided, usefully copackaged with specific unit dosage forms of dipyridamole, so as to facilitate the sequential sampling and mixture of both actives in the same syringe. Unit dosage forms can be prefilled syringes (see FIG. 4).

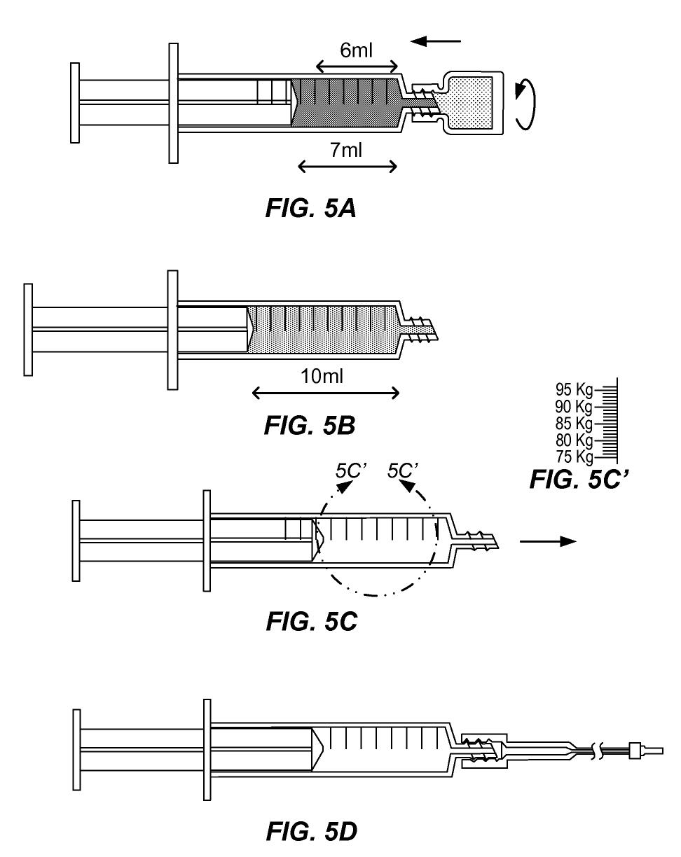

In other embodiments, the total volume of the dipyridamole unit dosage form is injected into the adenosine vial and the two are mixed so that only one sampling is required instead of two. Thus, as described below, in another aspect, the invention provides unit dosage forms of adenosine packaged so as to permit the sterile introduction of an appropriate volume of dipyridamole. Unit dosage forms of adenosine can also be prefilled syringes (FIG. 5).

In embodiments in which the two actives are combined in a single composition for concurrent administration, the volume to administer is usefully calculated based on the adenosine doses described herein.

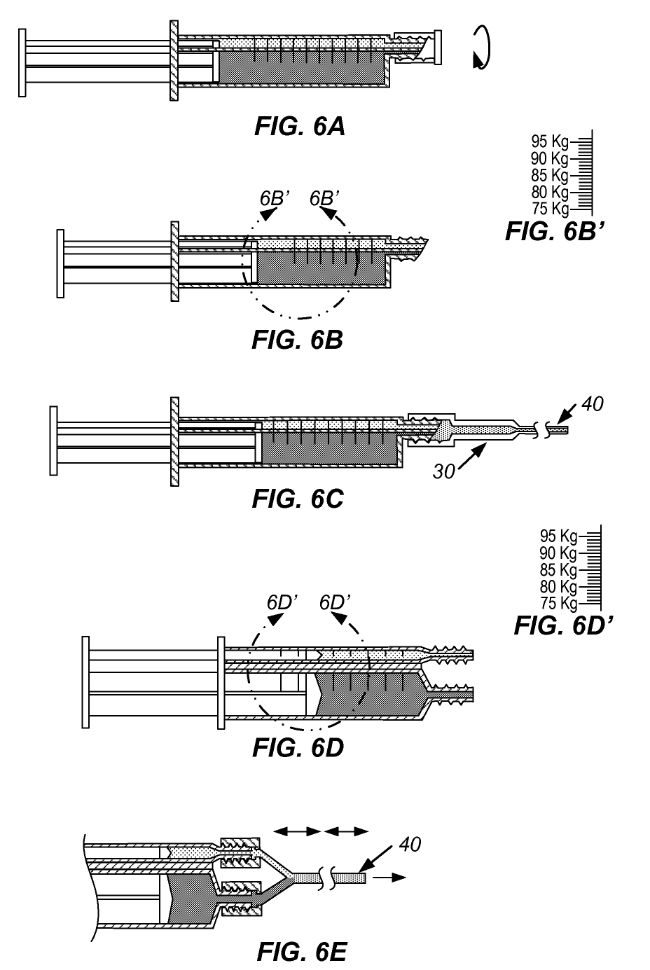

In other embodiments, dipyridamole and adenosine are concurrently administered from separate compositions. These separate compositions can be prefilled syringes. Usefully, the two agents may be introduced into the same infusion line using a Y connector (at the same dosages as set forth above) or a single connector specifically designed to mix the two products before they reach the venous line (see, e.g., FIG. 6).

In some concurrent administration embodiments, a very low priming dose of dipyridamole is administered prior to the concurrent infusion of adenosine and dipyridamole at reduced dosages (e.g., Example 4 below). Typically, this priming dose represents a fraction, in the 0.05-0.5 mg range, of the total dipyridamole dose to be administered into the venous line prior to the combination. It can be injected independently via a Y connector or administered using the separate administration mode with two syringes working in parallel and a specific connector that makes it possible to automatically deliver the priming dose as soon as concurrent infusion starts.

### 4.3. Methods of Pharmacological Stress Testing

Vasodilation that is achieved according to the above-described methods will often be used as a pharmacological stressor in cardiac stress tests. Accordingly, in certain embodiments, the methods further comprise the step of assessing cardiac function.

Any method suitable for assessing cardiac function in cardiac stress testing may be used.

In various embodiments, for example, assessing cardiac function includes use of one or more techniques selected from the group consisting of: electrocardiography, echography (M mode, two-dimensional, and three dimensional), echo-doppler (in particular transthoracic echo-doppler), cardiac imaging, including planar (conventional) scintigraphy, single photon emission computed tomography (SPECT), dynamic single photon emission computed tomography (D-SPECT™ Cardiac Scan), positron emission tomography (PET), radionuclide angiography (first pass and equilibrium studies utilizing, e.g., technetium-99 m-labeled red blood cells), nuclear magnetic resonance (NMR) imaging, myocardial perfusion contrast echocardiography, digital subtraction angiography (DSA), x-ray computed tomography (CINE CT) including high speed and ultra high speed CT scanning.

SPECT and PET present certain advantages, not least by providing images of the myocardial perfusion status, showing the presence or absence of reversible defects (ischemia), their location and their severity.

SPECT studies can be performed using any of the isotopes known to be suitable for such studies, such as thallium-201, technetium sestamibi, tetrofosmine. PET studies can be performed using any of the isotopes known to be suitable for perfusion studies, this including for example rubidium-82, Copper 62 PTSM [Copper-62-Pyruvaldehyde-bis-(⁴N-thiosemicarbazone)], nitrogen-13 (¹³N-ammonia), ¹⁵O-water (H2¹⁵0 Water), fluorine-18 (such as ¹⁸F-fluorodihydrorotenone or fluoromisonidazole) but also metabolic myocardial imaging studies with ¹⁸Fluorine-2-Deoxyglucose, carbon-11 (such as 1-[¹¹C] acetate, or C-11 palmitate), Nicotinic acid derivatives, Other isotopes are possible such as boron-11 etc.

Typically, isotope is injected during the infusion of adenosine, and imaging begins after the end of the infusion. In some embodiments, the isotope is administered no less than about 2 minutes after adenosine infusion has begun.

Echodoppler usefully presents a different advantage over SPECT and PET. It permits a fast and easy hemodynamic assessment of the coronary reserve for a specific coronary artery, which is another way to evaluate myocardial perfusion status. Myocardial perfusion contrast echography (Real-time MCE) avoids the use of isotopes and exposure to radiation. These techniques may include parenteral administration of an agent detectable by ultrasound techniques such as, but not only, hydrophobic drugs and/or polymers (e.g polyethylene glycol), in the form of microspheres or nanospheres and/or microbubbles made of gas including air or compositions comprising combinations of these agents (e.g. perflubutane polymers).

Ultra high speed CT scanners, such as the Somatom Definition Flash by Siemens, can scan the chest in less than one second. The technology not only reduces radiation exposure, but also facilitates the exploration of the beating heart without the requirement that the patient hold his or her breath during the exam. High speed and ultra high speed CT scans can be performed repeatedly at baseline and under stressed conditions for MPI studies.

In other embodiments, cardiac function, such as myocardial perfusion, is assessed using ultrasound detection of ultrasound contrast agents, such as ultrasound contrast agents with controlled fragility, as described in U.S. Pat. Nos. 6,776,761 and 6,193,951, the disclosures of which are incorporated herein by reference in their entireties.

### 4.4. Pharmaceutical Compositions

In one aspect, pharmaceutical compositions that are useful in the above-described methods are provided.

In typical embodiments, the pharmaceutical composition comprises adenosine and dipyridamole in an adenosine:dipyridamole (A:D) weight ratio of about 2:1 to about 10:1, with intermediate (including nonintegral) values permissible. In certain embodiments in which adenosine is intended to be administered at 70 μg/kg/min, the ratio is usefully about 7:1, 8:1, 9:1 and 10:1, with intermediate and nonintegral ratios permissible. In other embodiments, in which adenosine is to be administered at 50 μg/kg/min or less, A:D ratios are usefully about 2:1, 3:1, and 4:1, with intermediate and nonintegral ratios between 2:1 and 4:1 permissible. For certain clinical methods, the composition usefully comprises adenosine and dipyridamole at an A:D weight ratio of about 7:1.

In certain embodiments, the composition has a pH of less than about 4, such as less than about 3.9, 3.8, 3.7, 3.6, 3.5, 3.4, 3.3, even less than about 3.2.

In certain embodiments, the pharmaceutical composition is suitable for intravenous, intra-atrial, or intra-arterial infusion.

The composition may, for example, be in the form of a sterile, nonpyrogenic, fluid composition.

In typical fluid embodiments, the concentration of adenosine is at least about 1 mg/ml, 2 mg/ml, 3 mg/ml, 4 mg/ml and possibly 5 mg/ml, with intermediate, nonintegral, values permissible. These embodiments typically have a pH of about 3.5 to about 8. In other typical fluid embodiments with readily a lower pH (e.g., pH 2-3.5), adenosine concentration can be higher, even at least about 5 mg/ml, 6 mg/ml, 7 mg/ml, 8 mg/ml, 9 mg/ml and even 10 mg/ml, with intermediate, nonintegral, values permissible. In typical pharmaceutical composition embodiments, adenosine is present at a concentration of about 3 mg/ml, 4 mg/ml, 5 mg/ml, or 7 mg/ml.

In various fluid embodiments, the concentration of dipyridamole is at least about 0.1 mg/ml, and may usefully be as high as 4 mg/ml. The concentration may, in certain embodiments, be at least about 0.1 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1 mg/ml, or more, including, e.g., 1.1 mg/ml, 1.2 mg/ml, 1.3 mg/ml, 1.4 mg/ml, 1.5 mg/ml, 1.6 mg/ml, 1.7 mg/ml, 1.8 mg/ml, 1.9 mg/ml, 2 mg/ml, 2.1 mg/ml, 2.2 mg/ml, 2.3 mg/ml, or 2.4 mg/ml, 2.5 mg/ml, 3 mg/ml, 3.5 mg/ml, 4 mg/ml, with intermediate and nonintegral values permissible (e.g., 0.43, 0.57, 0.71, 0.86 mg/ml).

In certain embodiments, the composition comprises adenosine at a concentration of about 3 mg/ml, and dipyridamole at a concentration of about 0.375-0.428 mg/ml (A:D ratios of 8:1 and 7:1), which may be rounded to 0.38-0.43 mg/ml. In one embodiment, for example, the composition comprises adenosine at a concentration of about 3 mg/ml and dipyridamole at a concentration of about 0.43 mg/ml (ratio 7:1). In another embodiment, the composition comprises adenosine at a concentration of about 4 mg/ml and dipyridamole at a concentration of about 0.5-0.57 mg/ml (ratios of about 8:1 to 7:1). In another embodiment, the composition comprises adenosine at a concentration of about 5 mg/ml and dipyridamole at a concentration of about 0.62-0.71 mg/ml (ratios of 8:1 and 7:1). In another embodiment the composition comprises adenosine at a concentration of about 6 mg/ml and dipyridamole at a concentration of about 0.86 mg/ml (ratio 7:1). In another embodiment, the composition comprises adenosine at a concentration of about 7 mg/ml and dipyridamole at a concentration of about 1 mg/ml (ratio 7:1), and so on, up to adenosine concentrations as high as 10 mg/ml.

In some embodiments, the composition is dry, and suitable for reconstitution prior to infusion by addition of a sterile fluid into which both dipyridamole and adenosine are readily solubilized. Usefully, the composition comprises adenosine and dipyridamole in amounts suitable to permit reconstitution in the enclosing vessel to the adenosine and dipyridamole concentrations above-described.

Whether fluid or dry, the pharmaceutical composition may further comprise carriers and excipients suitable for intravenous, intra-atrial, or intra-arterial administration, as are well known in the art. Among such excipients are those used in currently approved dipyridamole and adenosine compositions, such as tartaric acid, hydrochloric acid and polyethylene glycol (macrogol 600). Others are permissible, such as, for example, mannitol. See, http://www.adenosin.com/en/en_SPC_{—}05.pdf (Item Development AB, 2005), incorporated herein by reference. See also, Remington: The Science and Practice of Pharmacy, 21^{st} ed. (2005), Lippincott Williams & Wilkins (ISBN: 0781746736), incorporated herein by reference.

The compositions may further comprise additional actives, and in some embodiments, may further comprise contrast agents, including ultrasound and MRI contrast agents.

In embodiments intended for continuous intravenous infusion in the methods above-described, adenosine is typically present in the pharmaceutical composition at a concentration, or in a weight amount, that permits adenosine to be infused at a rate between about 35 μg/kg/min to about 100 μg/kg/min.

In some of these embodiments, adenosine is present in an amount that permits infusion at a rate of at least about 35 μg/kg/min, at least about 40 μg/kg/min, at least about 45 μg/kg/min, at least about 50 μg/kg/min, at least about 55 μg/kg/min, at least about 60 μg/kg/min, at least about 65 μg/kg/min, at least about 70 μg/kg/min, at least about 75 μg/kg/min, at least about 80 μg/kg/min, at least about 85 μg/kg/min, at least about 90 μg/kg/min, at least about 95 μg/kg/min, and at least about 100 μg/kg/min, with intermediate and nonintegral values permissible.

In some embodiments, adenosine is present in the composition in an amount that permits infusion at a rate of no more than about 100 μg/kg/min, no more than about 95 μg/kg/min, no more than about 90 μg/kg/min, no more than about 85 μg/kg/min, no more than about 80 μg/kg/min, no more than about 75 μg/kg/min, no more than about 70 μg/kg/min, no more than about 65 μg/kg/min, no more than about 60 μg/kg/min, no more than about 55 μg/kg/min, no more than about 50 μg/kg/min, no more than about 45 μg/kg/min, no more than about 40 μg/kg/min, no more than about 35 μg/kg/min, with intermediate and nonintegral values permissible.

In embodiments intended for continuous intravenous infusion, dipyridamole is typically present in the pharmaceutical composition at a concentration, or in a weight amount, that permits dipyridamole to be infused at a rate between about 3.5 μg/kg/min to 50 μg/kg/min.

In some of these embodiments, dipyridamole is present in an amount that permits infusion at a rate of at least about 3.5 μg/kg/min, at least about 4 μg/kg/min, at least about 5 μg/kg/min, at least about 6 μg/kg/min, at least about 7 μg/kg/min, at least about 7.5 μg/kg/min, at least about 8 μg/kg/min, at least about 8.75 μg/kg/min, at least about 9 μg/kg/min, at least about 9.25 μg/kg/min, at least about 9.50 μg/kg/min at least about 10 μg/kg/min, at least about 11 μg/kg/min, at least about 11.25 μg/kg/min, at least about 12 μg/kg/min, at least about 12.5 μg/kg/min, at least about 13 μg/kg/min, at least about 13.75 μg/kg/min, at least about 14 μg/kg/min, at least about 15 μg/kg/min, at least about 16 μg/kg/min, at least about 16.25 μg/kg/min, at least about 17 μg/kg/min, and at least about 17.5 μg/kg/min, at least about 18 μg/kg/min, at least about 19 μg/kg/min, at least about 20 μg/kg/min, at least about 21 μg/kg/min, at least about 22 μg/kg/min, at least about 23 μg/kg/min, at least about 24 μg/kg/min, at least about 25 μg/kg/min, at least about 26 μg/kg/min, at least about 27 μg/kg/min, at least about 28 μg/kg/min, at least about 29 μg/kg/min, at least about 30 μg/kg/min, at least about 31 μg/kg/min, at least about 32 μg/kg/min, at least about 33 μg/kg/min, at least about 34 μg/kg/min, at least about 35 μg/kg/min, at least about 36 μg/kg/min, at least about 37 μg/kg/min, at least about 38 μg/kg/min, at least about 39 μg/kg/min, at least about 40 μg/kg/min, at least about 41 μg/kg/min, at least about 42 μg/kg/min, at least about 43 μg/kg/min, at least about 44 μg/kg/min, at least about 45 μg/kg/min, at least about 46 μg/kg/min, at least about 47 μg/kg/min, at least about 48 μg/kg/min, at least about 49 μg/kg/min, at least about 50 μg/kg/min, with intermediate and nonintegral values permissible.

In some embodiments, dipyridamole is present in the composition in an amount that permits intravenous infusion at a rate of no more than about 50 μg/kg/min, no more than about 49 μg/kg/min, no more than about 48 μg/kg/min, no more than about 47 μg/kg/min, no more than about 45 μg/kg/min, no more than about 44 μg/kg/min, no more than about 43 μg/kg/min, no more than about 42 μg/kg/min, no more than about 41 μg/kg/min, no more than about 40 μg/kg/min, no more than about 39 μg/kg/min, no more than about 38 μg/kg/min, no more than about 37 μg/kg/min, no more than about 36 μg/kg/min, no more than about 35 μg/kg/min, no more than about 34 μg/kg/min, no more than about 33 μg/kg/min, no more than about 32 μg/kg/min, no more than about 31 μg/kg/min, no more than about 30 μg/kg/min, of no more than about 29 μg/kg/min, no more than about 28 μg/kg/min, no more than about 27 μg/kg/min, no more than about 26 μg/kg/min, no more than about 25 μg/kg/min, no more than about 24 μg/kg/min, no more than about 23 μg/kg/min, no more than about 22 μg/kg/min, no more than about 21 μg/kg/min, no more than about 20 μg/kg/min, no more than about 19 μg/kg/min, no more than about 18 μg/kg/min, no more than about 17.5 μg/kg/min, no more than about 17 μg/kg/min, no more than about 16.25 μg/kg/min, no more than about 16 μg/kg/min, no more than about 15 μg/kg/min, no more than about 14 μg/kg/min, no more than about 13.75 μg/kg/min, no more than about 13 μg/kg/min, no more than about 12.5 μg/kg/min, no more than about 12 μg/kg/min, no more than about 11.25 μg/kg/min, no more than about 11 μg/kg/min, no more than about 10 μg/kg/min, no more than about 9.25 μg/kg/min, no more than about 9.50 μg/kg/min no more than about 9 μg/kg/min, no more than about 8.75 μg/kg/min, no more than about 8 μg/kg/min, no more than about 7.5 μg/kg/min, no more than about 7 μg/kg/min, no more than about 6 μg/kg/min, no more than about 5 μg/kg/min, no more than about 4 μg/kg/min, no more than about 3.5 μg/kg/min, with intermediate and nonintegral values permissible.

### 4.5. Unit Dosage Forms

4.5.1. Adenosine:Dipyridamole Combined Compositions

The pharmaceutical compositions described herein are usefully packaged in a unit dosage form adapted for use in the methods described herein.

In some embodiments, the pharmaceutical composition is in the form of a liquid suitable for parenteral infusion and the composition may, for example, be packaged in volumes ranging from 2 to 50 ml. Convenient unit dosage forms contain 2 to 14 ml, typically 2, 3, 4, 5, 6, 7, 8, or 14 ml. Unit dosage forms containing volumes as low as 1 ml, and unit dosage forms containing higher volumes, such as 15 or 20 ml, are also possible. Intermediate and nonintegral volumes are permissible.

Table 1 provides examples of unit dosage form embodiments of the adenosine dipyridamole pharmaceutical compositions herein described.

TABLE 1

| | | Total amount | Maximal patient | Maximal patient | Total amount |
|---|---|---|---|---|---|
| Adenosine | | of adenosine | weight (in kgs) | weight (in kgs) | of dipyridamole |
| concentration | Volume | per dosage | covered when | covered when | (A:D ratio 7:1) |
| (mg/ml) | (ml) | unit (mg) | infusion time is of 4 mins | infusion time is of 3 mins | (mg) |
| 3 | 7 | 21 | 75 | 100 | 3 |
| | 14 | 42 | 150 | 200 | 6 |
| 4 | 7 | 28 | 100 | | 4 |
| | 14 | 56 | 200 | | 8 |
| 5 | 7 | 35 | 125 | | 5 |
| 6 | 7 | 42 | 150 | 200 | 6 |
| 7 | 2 | 14 | 50 | | 2 |
| | 3 | 21 | 75 | 100 | 3 |
| | 4 | 28 | 100 | 133 | 4 |
| | 5 | 35 | 125 | | 5 |
| | 6 | 42 | 150 | 200 | 6 |
| | 8 | 56 | 200 | | 8 |

Thus, in some embodiments, the unit dosage form usefully contains 14 mg of adenosine and 2 mg of dipyridamole in 2 ml; 21 mg of adenosine and 3 mg of dipyridamole in 3 or 7 ml; 28 mg of adenosine and 4 mg of dipyridamole in 4 or 7 ml; 35 mg of adenosine and 5 mg of dipyridamole in 5 or 7 ml; 42 mg of adenosine and 6 mg of dipyridamole in 6 or 7 or 14 ml; 56 mg of adenosine and 8 mg of dipyridamole in 8 or 14 ml.

In some embodiments, the unit dosage forms can be prefilled syringes (see, e.g., FIGS. 4-6). In a variety of embodiments, prefilled syringes have a total capacity as described above of 5, 10, 15, 20 or even 30 ml, allowing addition of saline to the dose if needed to adjust the pH or provide an administration volume more readily programmable on an electric pump or similar automated delivery device. Use of saline may be called for where the composition has an acidic pH of about 2 to 4.5.

In certain embodiments, the unit dosage forms are vials as described in Table 1 and the pharmaceutical compositions are sampled using empty syringes of standard size of 5, 10, 15, 20, and even 30 ml total capacity.

Whether pre-filled, or to be filled by sampling one or more vials, syringes for use in the methods of the present invention may be usefully labeled with a weight graduation scale to facilitate weight-adjustment before administration.

Accordingly, in one aspect of the present invention, syringe labels are provided with weight graduation scales and the corresponding volume of a pharmaceutical composition comprising an active to be administered based on a patient's weight. These labels permit rapid and accurate calculation of the dose to be administered based on patient weight, reducing the likelihood of a dosing error. Using the labels, dose can be directly adjusted according to the patient's weight by moving the syringe plunger to the appropriate weight on the label affixed to the syringe barrel. In some embodiments, the unit of weight is a kilogram. In some embodiments, the unit of weight is a pound.

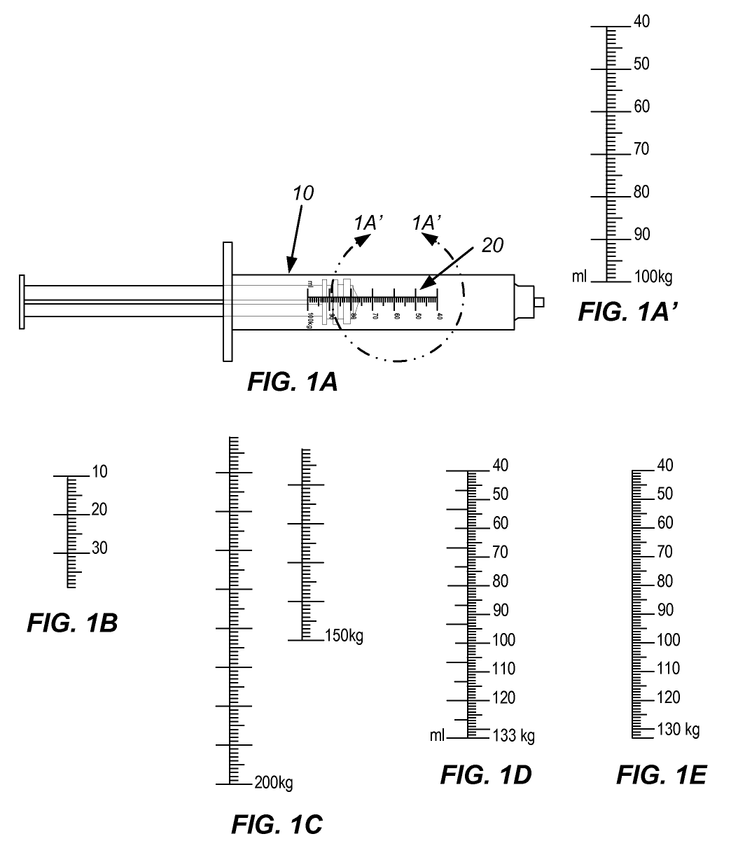

Labels comprise weight graduations for dosing and can be tailored based on desired infusion time, concentration of active(s) in the pharmaceutical composition, and administration volume. See FIGS. 1 and 4. For example, if infusion time is 4 minutes and the composition to be administered comprises 28 mg adenosine and 4 mg dipyridamole in a 4 ml volume, a 10-ml prefilled syringe can be labeled with a graduation scale where 1 kilogram=0.04 ml. In this embodiment, a 4 ml injection provides the dose appropriate for a patient weighing 100 kgs, or 4 ml=100 kg. Similarly, in an embodiment where the same composition is provided in a volume of 7 ml as described in Table 1, a prefilled syringe of 15 or 20 ml is recommended with a graduation scale where a one kilogram interval corresponds to 0.07 ml and 7 ml=100 kg. In one embodiment, where the composition comprises 35 mg adenosine with 5 mg dipyridamole in a 7 ml volume, a prefilled syringe of 15 or 20 ml is used with a graduation scale such as a one kilogram interval is equivalent to 0.056 ml and 7 ml=100 kg. Given the present disclosure, one of skill in the art can readily make labels with appropriate weight and volume graduations for any of the compositions or dosage forms described herein. Scales can be expressed in any unit that is known to those of skill in the art. For example, weight units including, but not limited to, kilograms or pounds may be used.

As yet another example, if infusion time is 3 minutes, the weight graduation scale is modified accordingly. For example, labels can be provided in which kilogram intervals equivalent to ¾ (×0.75) the value of those described above, or a 1 kilogram interval corresponds to 0.03 ml (where the composition comprises 28 mg adenosine and 4 mg dipyridamole in 4 ml) or 0.0525 ml (where the composition comprises 28 mg adenosine and 4 mg dipyridamole in 7 ml) or 0.042 ml (where the composition comprises 35 mg adenosine and 5 mg dipyridamole in 7 ml).

In some embodiments, the weight graduation scale is in kilograms and the scale can range from 10 or 40 kilograms up to 100 or 125 or 133 or 150 or 166 or even 200 kilograms depending on the syringe capacity. In some embodiments, the milliliter per kg equivalence can range typically from 0.01 ml=1 kg to 0.2 ml=1 kg depending on the dose, the volume of liquid in the syringe, and the recommended infusion time. In some embodiments, the graduation scale is such that a one kilogram interval can equal 0.03 ml, or 0.04 ml or 0.042 ml, or 0.0525 ml, or 0.056 ml, or 0.07 ml, according to needs and the features of the syringe used. In some embodiments, syringes are provided with a weight graduation scale in pounds.

The container for unit dosage embodiments is typically adapted for use with standard intravenous infusion sets, syringe pumps and adapted connectors.

Whether liquid or dry, the unit dosage form is typically sterile and nonpyrogenic.

4.5.2. Dipyridamole Unit Dosage Forms

In typical embodiments of the methods herein described, dipyridamole is administered at about 5% of the dose at which it is currently administered as a single agent. Accordingly, in one aspect, the invention provides novel unit dosage forms of dipyridamole.

In some embodiments, dipyridamole unit dosages forms are provided in prefilled syringes. Prefilled syringes of 1 or 2 ml with compositions comprising dipyridamole at about 2 to about 4 mg/ml and of a length sufficient to allow a clear reading of graduation marks allow for immediate dose adjustment according to patient weight and can be used without predilution in saline despite the acidity of dipyridamole's medium (pH=2-3.6). In certain embodiments, the final dose can be injected before adenosine administration directly into a venous line via a Y connector provided a long enough line (e.g. about 100 cms) filled with solution to ensure instantaneous dilution of dipyridamole. In this example, the recommended ratio of dipyridamole to the buffered solution is 1:2. In some embodiments, an extension set with an inner volume of at least 2 ml and possibly 4 ml is provided along with the prefilled syringe allowing sufficient dipyridamole dilution. In some embodiments, unit dosage forms or prefilled syringes of 3 mg/ml, 4 mg/ml, 6 mg/2 ml or 8 mg/2 ml) are provided.

Dipyridamole unit dosage forms (dipyridamole concentrations of 2 mg/ml, 3 mg/ml, 4 mg/ml, 6 mg/2 ml, or 8 mg/2 ml) are also convenient for concurrent administration with adenosine. In some embodiments, the entire dipyridamole unit dose is mixed into the adenosine unit dose, prior to administration (see FIG. 5). In some embodiments, dipyridamole is administered concurrently with adenosine using separate syringes with the each of the two syringes' open ends in fluid communication with, and the syringe contents flowing into, the same connector, where the two products are mixed before reaching the venous line as a single composition (see FIG. 6).

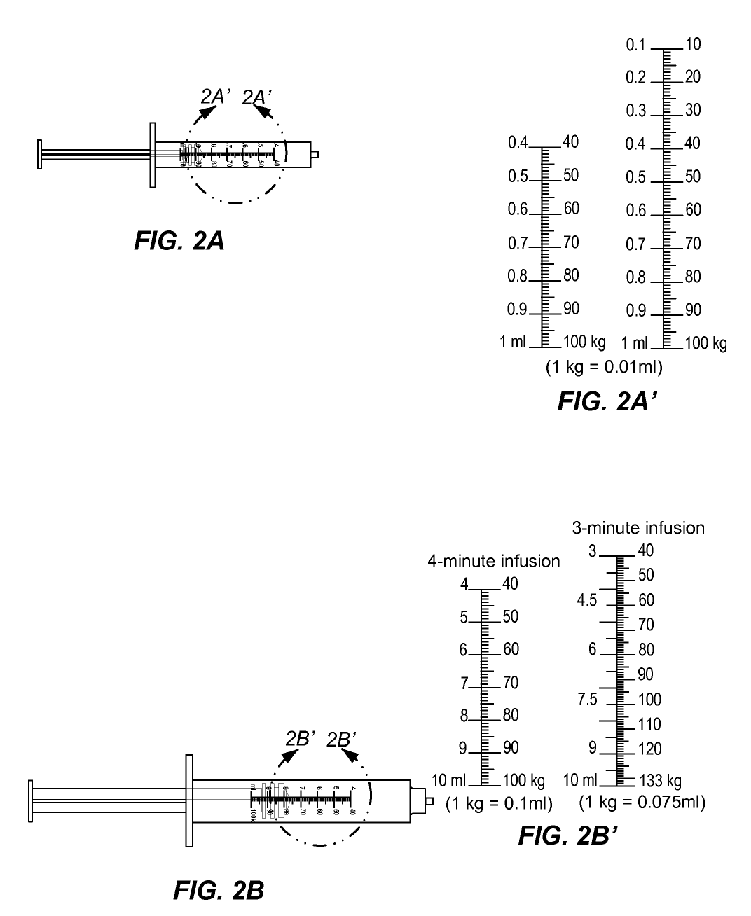

In various embodiments, a label for synthetic (plastic, resin, polymer, or equivalent) or glass syringes prefilled with 1 or 2 ml of dipyridamole alone (typically 4 mg/ml) with kilo graduation marks on the barrel is designed providing 0.01 ml intervals per kilogram (see example 7 and FIG. 2A).

Specific dipyridamole unit dosage forms of 1 mg in 1 ml that can be prefilled syringes are particularly useful when a priming dose is injected into the venous line independently from any other system.

4.5.3. Adenosine Unit Dosage Forms

In some embodiments, dipyridamole and adenosine are sampled from separate unit dosage forms, and mixed in the same syringe. In these embodiments, convenient adenosine unit dosage forms are 14 mg of adenosine in 7 ml, or 28 mg of adenosine in 7 ml and 56 mg of adenosine in 14 ml (4 mg/ml adenosine) or 21 mg of adenosine in 7 ml and 42 mg of adenosine in 14 ml (3 mg/ml adenosine). Possibly also 35 mg of adenosine in 15 ml. The syringe employed for sampling can be of standard 10, 15 or 20 ml and even 30 ml capacity so as to cope with all syringe pump systems and be usefully custom scaled, as above-described, with kilo, pound, or other, weight graduation marks. In certain embodiments, the adenosine unit dosage forms are prefilled syringes.

In some specific embodiments convenient unit dosage forms are 21 mg of adenosine in 10 ml, or 42 mg in 20 ml (adenosine concentration=2.1 mg/ml), 28 mg of adenosine in 10 ml or 56 mg in 20 ml (adenosine concentration=2.8 mg/ml). Unit dosage forms of 14 mg adenosine in 10 ml and 35 mg adenosine in 10 ml are also possible. Unit dosage forms are preferably prefilled syringes so as to avoid the sampling phase. In these embodiments saline may be added after adjustment of the dose into the syringe to reconstitute 10 or 20 ml prior to infusion if adenosine is infused via an electric pump. However in some cases adenosine prefilled syringes can be administered manually directly into the venous line as a 2 to 3 minute bolus-infusion without addition of saline.

In some embodiments, the unit dosage form comprises a pre-filled syringe comprising 60 mg adenosine in a volume of 20 ml. In some embodiments, the unit dosage form comprises a pre-filled syringe comprising 90 mg adenosine in a volume of 30 ml.

Regardless of their volume, and irrespective of whether prefilled or empty, to be filled by sampling, the syringe barrel in various embodiments is usefully inscribed with scales with patient weight graduation marks in order to facilitate dose adjustment. Automated labeling systems, e.g., systems using glue to fix and wrap the label around the barrel of 5, 10, 15, 20, 30, 40, or 50 ml syringes (as well as around syringes of non standard capacity), or shrink-wrap label techniques are well known industrial procedures by those skilled in the art. In other embodiments, gradations can be molded directly into the barrel.

Depending on the intended or desired infusion time and the volume of liquid in the syringe, different labels with different weight graduation scales can be made.

For example, if infusion time is 4 minutes and the composition comprises 28 mg of adenosine prefilled in a 10-ml syringe, the graduation scale is similar to the one shown in FIG. 1 with an interval of 1 kilogram=0.1 ml wherein 10 ml equals 100 kgs, 6 ml=60 kgs, and so on. If the intended infusion time is of 3 minutes, the graduation scale will be similar to the other scale shown in FIG. 1 with an interval of 1 kilogram=0.075 ml wherein 10 ml is equivalent to 133 kgs, 9 ml to 120 kgs, 7.5 ml to 100 kg, and so on. If infusion time is of 4 minutes and the composition made of 35 mg adenosine in 10 ml and prefilled in a syringe of 10 or 15 ml capacity, the 1 kilogram interval is =0.08 ml. If infusion time is of 3 minutes, the kilogram interval is =0.06 ml. For an infusion time of 3 minutes, the kilogram interval is equivalent to 0.09 ml when the preparation is made of 35 mg adenosine in 15 ml and to 0.12 ml when 35 mg adenosine are in a 20 ml volume. If the composition comprises 21 mg of adenosine prefilled in a 10-ml syringe and an infusion time of 3 minutes is desired, the graduation scale is similar to the one shown in FIG. 2, where 1 kilogram=0.1 ml, and, for example, 6 ml is the dose for 60 kgs, and 10 ml is the dose for 100 kgs. If the composition comprises 21 mg of adenosine prefilled in a 10 ml syringe and an infusion time of 2 minutes is desired, the graduation scale is usefully such that 1 kg=0.14 ml, and, for example, 10 ml is the dose for 150 kg.

As described above, in some embodiments, dipyridamole and adenosine are usefully provided in separate pharmaceutical compositions, and administered sequentially. However in other embodiments there are combined prior to administration. In some of these embodiments, a dipyridamole composition is usefully introduced into a unit dose of adenosine, and the combined composition then administered.

Thus, unit dosage forms of adenosine are provided, in which adenosine is formulated in sterile fluid composition, and in which the dose packaging permits sterile introduction of a second fluid in a volume at least 15% that of the adenosine composition (see, e.g., FIG. 5).

Adenosine may be present at any of the concentrations at which it is present in the pharmaceutical compositions above-described but preferably from 1 mg/ml to 5 mg/ml—either as directly packaged, or as thereafter will achieved upon introduction of an appropriate amount of dipyridamole composition.

For example, in one embodiment, an adenosine unit dosage form contains 21 mg adenosine in 6 ml (21 mg/6 ml). This will reconstitute to a desired 21 mg adenosine/7 ml (3 mg/ml adenosine) composition upon introduction of 1 ml of 3 mg/ml dipyridamole (e.g., the entirety of a unit dose of dipyridamole containing 1 ml dipyridamole at 3 mg/ml). In another embodiment, an adenosine unit dosage form contains 42 mg/12 ml. This will reconstitute to a desired 42 mg/14 ml (3 mg/ml) adenosine upon introduction of a 6 mg/2 ml dipyridamole unit dose. In another embodiment, the adenosine unit dosage form contains 28 mg adenosine/6 ml, which will reconstitute to 28 mg/7 ml (4 mg/ml adenosine) upon introduction of a 4 mg/ml dipyridamole unit dose. In another embodiment, the adenosine unit dosage form contains 56 mg adenosine/12 ml, which will reconstitute to 56 mg/14 ml (4 mg/ml adenosine) upon introduction of an 8 mg/2 ml dipyridamole unit dose. In another embodiment, the adenosine unit dosage form contains 35 mg adenosine/14 ml, which will reconstitute to 35 mg/15 ml (3 mg/ml adenosine) upon introduction of an 5 mg/l ml dipyridamole unit dose. The following Table 2 summarizes exemplary unit dosage forms.

TABLE 2

| | | | | | | | Maximal |
|---|---|---|---|---|---|---|---|
| | | | | | | | patient's |
| | | Volume (ml) | | | Total | | weight |
| | | of | | | amount | | covered by |
| | | dipyridamole | Final | | of | Total | the |
| Initial | | solution | adenosine | | adenosine | amount of | composition |
| adenosine | Initial | added to | vial (ml) | Final | per unit | dipyridamole | (kgs) when |
| vial | adenosine | each | volume after | adenosine | dosage | (A:D ratio | infusion |
| volume | concentration | adenosine | addition of | concentration | form | 7:1) | time is |
| (ml) | (mg/ml) | vial | dipyridamole | (mg/ml) | (mg) | (mg) | 4 mins |
| 3 | 4.66 | 1 | 4 | 3.5 | 14 | 2 | 50 |
| 6 | 3.5 | 1 | 7 | 3 | 21 | 3 | 75 |
| 6 | 4.66 | 1 | 7 | 4 | 28 | 4 | 100 |
| 12 | 3.5 | 2 | 14 | 3 | 42 | 6 | 150 |
| 12 | 4.66 | 2 | 14 | 4 | 56 | 8 | 200 |
| 14 | 2.50 | 1 | 15 | 3 | 35 | 5 | 125 |

In certain embodiments the total capacity of prefilled syringes comprising unit doses as described in table 2 is of 5, 10, 15, 20 or 30 ml, which are standard sizes compatible with various types of electrical pumps and permitting, if necessary, the addition of saline.

In other embodiments dipyridamole and adenosine are concurrently given from separate unit dosage forms or prefilled syringes that deliver the two drugs in parallel into the same venous line. In these embodiments, convenient adenosine unit dosage forms are the same as those described above.

Typically, dosing and sampling are determined according to adenosine tables.

4.6. Kits

In another aspect, kits are provided in which one or more unit dosage forms of dipyridamole, such as those above-described, are packaged with one or more unit dosage forms of adenosine, such as those above-described. Typically, the kit will comprise an equal number of dipyridamole doses and adenosine doses.

In some embodiments, the unit dosage of dipyridamole is a prefilled syringe containing for example 3 mg or 4 mg or 6 mg or 8 mg of active ingredient and the adenosine unit dosage form another prefilled syringe with for example 21 mg or 28 mg or 42 mg or 56 mg of active ingredient so as to respect the preferred 1:7 ratio between the two products.

In certain embodiments these two prefilled syringes, although separated, can share the same plunger (or have these two pieces joined together) and can usefully present two open ends that are in fluid communication with, and the contents flowable into, the same connector for the mixing of the two products before they reach the extension set (e.g., venous line).

In some embodiments, the unit dose of dipyridamole is packaged in a pre-packed vial, and the adenosine unit dose is packaged as a prefilled syringe with an injection port, such as a septum, permitting sterile introduction of dipyridamole into the adenosine dose.

In some embodiments, the unit dosage form is a vial or a prefilled syringe containing both adenosine and dipyridamole as described above.

In certain embodiments wherein adenosine and dipyridamole are administered concurrently and preceded by a dipyridamole priming dose, a 1 mg in 1 ml dipyridamole prefilled syringe is provided in the kit.

In various embodiments, the kit further includes one or more of an adenosine dosage table, one or more needles, diluent (e.g., saline), appropriate connectors and infusion sets of at least 2 ml in total volume, preferably 4 ml.

### 5. EXAMPLES

### 5.1. Example 1

### Sequential Administration—Assessment of the Coronary Vasodilation Induced by the Combination by Transthoracic Doppler-Echography

The effects of administering sequentially dipyridamole and adenosine intravenously as a pharmacological stressor were compared to the effects of administering adenosine alone in 40 consecutive patients suffering from ischemic heart disease. In the combined administration, each of the two agents was administered at a dosage lower than its clinically preferred dosage when used as a single agent for myocardial perfusion imaging. Effects were measured using noninvasive transthoracic doppler echocardiography (TTDE).

Primary efficacy end-points were peak and mean diastolic flow velocities (measured as reflecting coronary blood flow values). The secondary end-point was patient tolerance to the procedure. The protocol was designed as follows.

Forty (40) consecutive patients suffering from ischemic heart disease were enrolled. Each patient served as his own control.

Adenosine was administered by IV infusion for three minutes at the standard single-agent infusion rate of 140 μg/kg/min.

After a stabilization period of five minutes, patients then received an IV injection of dipyridamole at a total dose of either 23 μg/kg, 28 μg/kg, or 35 μg/kg, administered as a bolus over about 20-30 seconds. Total doses are between about 4-6% of the lowest single-agent total dose of dipyridamole (i.e., 0.56 mg/kg, infused over a total of 4 minutes). Preliminary dose ranging studies at lower doses of dipyridamole (10, 14 and 18 μg/kg) showed no effect.

The bolus injection of dipyridamole was followed immediately by an IV infusion of adenosine at 70 μg/kg/min for 3 minutes. This dose is half the standard single-agent dosage rate of 140 μg/kg/min.

Blood flow velocity was measured in the left anterior descending coronary artery (LAD) at four time points: (i) before initial adenosine infusion (spontaneous flow at rest), (ii) during the initial 140 μg/kg/min adenosine infusion, (iii) before the sequential administration of dipyridamole and adenosine (during the stabilization period), and (iv) during the 70 μg/kg/min adenosine infusion, subsequent to dipyridamole bolus injection.

Results are given in Tables 3-6. Abbreviations used in the table are defined below:

ADE: adenosine alone at 140 μg/kg/min

SC: sequential combination of dipyridamole followed by adenosine at 70 μg/kg/min

PV: peak velocity (cm/sec)

MV: mean velocity (cm/sec)

max: velocity under stress conditions

min: velocity at rest (under basal conditions)

( ): standard deviation

D %: velocity differential (peak or mean), as percentage of maximum peak or mean velocity

Table 3 presents results comparing dipyridamole 28 μg/kg IV bolus (over 20-30 seconds) followed by adenosine infusion at 70 μg/kg/min (“DIP5” sequential combination) as compared to adenosine infusion alone at the standard single-agent dose of 140 μg/kg/min, in 30 patients.

TABLE 3

| | Peak | | Peak | | Mean | | Mean | |
|---|---|---|---|---|---|---|---|---|
| | velocity | | velocity | | velocity | | velocity | |
| | ADE | | SC | | ADE | | SC | |
| | Max | Min | Max | Min | Max | Min | Max | Min |
| Mean (s.d.) | 82.6 | 30.6 | 81 | 30.6 | 61.4 | 23.1 | 60.2 | 22.9 |
| | (20.7) | (8.5) | (21) | (9.6) | (15.1) | (6.1) | (15.3) | (7.3) |

| | | |
|---|---|---|
| Max velocity differential | PV: 82.6 − 81 = 1.6 | MV: 61.4 − 60.2 = 1.2 |
| Max velocity differential (D %) | 1.9% | 1.9% |
| P value | 0.217 | 0.201 |

Table 4 presents results comparing dipyridamole (35 μg/kg) bolus (administered over 20-30 seconds), followed by adenosine, administered by infusion at a rate of 70 μg/kg/min (“DIP4” sequential combination) as compared to adenosine infusion alone at the standard single-agent dose of 140 μg/kg/min, in 5 patients.

TABLE 4

| | Peak | | Peak | | Mean | | Mean | |
|---|---|---|---|---|---|---|---|---|
| | velocity | | velocity | | velocity | | velocity | |
| | ADE | | SC | | ADE | | SC | |
| | Max | Min | Max | Min | Max | Min | Max | Min |
| Mean (s.d.) | 80 | 28 | 78.8 | 28 | 60.4 | 22.2 | 57.8 | 23 |
| | (22.3) | (7.4) | (15.3) | (7.7) | (15.5) | (5) | (14.4) | (5.7) |

| | | |
|---|---|---|
| Max velocity differential | PV: 80 − 78.8 = 1.2 | MV: 60.4 − 57.8 = 2.6 |
| Max velocity differential | 1.5% | 4.3% |
| (D %) | | |
| P value | 0.990 | 0.448 |

Table 5 presents results comparing dipyridamole (23 μg/kg), administered as a bolus over 20-30 seconds, followed by adenosine, administered by infusion at a rate of 70 μg/kg/min (“DIP6” sequential combination) as compared to adenosine infusion alone at the standard single-agent dose of 140 μg/kg/min, in 5 patients.

TABLE 5

| | Peak | | Peak | | Mean | | Mean | |
|---|---|---|---|---|---|---|---|---|
| | velocity | | velocity | | velocity | | velocity | |
| | ADE | | SC | | ADE | | SC | |
| | Max | Min | Max | Min | Max | Min | Max | Min |
| Mean (s.d.) | 107 | 37.6 | 105.4 | 38.6 | 80.2 | 28.2 | 79.2 | 30 |
| | (36.5) | (12.1) | (34.3) | (16.5) | (25.4) | (11.1) | (24.2) | (14.7) |

| | | |
|---|---|---|
| Max velocity | PV: 107 − 105.4 = 1.6 | MV: 80.2 − 79.2 = 1 |
| differential | | |
| Max velocity | 1.5% | 1.2% |
| differential (D %) | | |
| P value | 0.875 | 0.830 |

Table 6 presents results cumulated from all 40 patients:

TABLE 6

| | Peak | | Peak | | Mean | | Mean | |
|---|---|---|---|---|---|---|---|---|
| | velocity | | velocity | | velocity | | velocity | |
| | ADE | | SC | | ADE | | SC | |
| | Max | Min | Max | Min | Max | Min | Max | Min |
| Mean (s.d.) | 85.3 | 31.2 | 83.8 | 31.3 | 63.6 | 23.7 | 62.3 | 23.7 |
| | (24) | (9) | (23.3) | (10.5) | (17.3) | (6.8) | (17.3) | (8.4) |

| | | |
|---|---|---|
| Max velocity differential | PV: 85.3 − 83.8 = 1.5 | MV: 63.6 − 62.3 = 1.3 |
| Max velocity differential | 1.75% | 2% |
| (D %) | | |
| P value | 0.314 | 0.109 |

The measured blood flow velocities, whether peak or mean, were 3 to 4% lower, in absolute values, than those of adenosine alone (see Tables 3 to 6). However, these differences were not statistically significant (all P values >0.05): there was no statistical difference between the standard treatment—infusion of adenosine alone at 140 μg/kg/min—and sequential bolus administration of dipyridamole (at 4-6% its typical single-agent total dose) followed by adenosine at 70 μg/kg/min, whether assessed separately for each of the three tested dipyridamole doses (Tables 3-5), or cumulated across all dipyridamole doses (Table 6).

Tolerance: occurrence and severity of adverse events

Table 7 shows the number and frequency of occurrence among all 40 patients of the three adverse events most commonly observed in clinical practice upon administration of adenosine alone at 140 μg/kg/min: chest pain, dyspnea, and flushing.

TABLE 7

| | ADE | All SC (DIP 4/5/6) | |
|---|---|---|---|
| | # patients | # patients | |
| | reporting events | reporting events | |
| Adverse event | (frequency) | (frequency) | % reduction |
| Chest pain | 9 (22.5%) | 5 (12.5%) | −44% |
| Dyspnea | 20 (50%) | 18 (45%) | slight |
| Flushing | 21 (52.5%) | 17 (42.5%) | slight |

Table 8. Cumulative results on the severity of the three subjective symptoms of the composite (chest pain, dyspnea, flushing) rated from 0 to 30. Cumulative results on the severity of the three subjective symptoms of the composite (chest pain, dyspnea, flushing) rated from 0 to 30.

TABLE 8

| | Adenosine alone | SC |
|---|---|---|
| DIP4 (n = 5) | | |
| Chest pain | 1.2 ± 2.68 | 0 ± 0 |
| Dyspnea | 1.4 ± 1.95 | 1.2 ± 1.79 |
| Flushing | 2.9 ± 2.88 | 0.8 ± 1.79 |
| Composite index | 5.5 ± 2.12 | 2 ± 2 |
| DIP5 (n = 30) | | |
| Chest pain | 1.12 ± 1.95 | 0.5 ± 1.28 |
| Dyspnea | 2.33 ± 2.59 | 1.87 ± 2.31 |
| Flushing | 2.35 ± 2.53 | 1.82 ± 2.35 |
| Composite index | 5.8 ± 3.44 | 4.18 ± 3.39 |
| DIP6 (n = 5) | | |
| Chest pain | 0 ± 0 | 0 ± 0 |
| Dyspnea | 5 ± 3.08 | 3 ± 3.08 |
| Flushing | 3.2 ± 3.42 | 1.4 ± 2.61 |
| Composite index | 8.2 ± 1.09 | 4.4 ± 2.41 |
| TOTAL (n = 40) | | |
| Chest pain | 0.99 ± 1.93 | 0.37 ± 1.12 |
| Dyspnea | 2.55 ± 2.71 | 1.92 ± 2.34 |
| Flushing | 2.52 ± 2.63 | 1.64 ± 2.29 |
| Composite index | 6.06 ± 3.17 | 3.94 ± 3.18 |

Table 9 presents cumulative statistical results on the severity of the three subjective symptoms of the composite (chest pain, dyspnea, flushing) rated from 0 to 30.

TABLE 9

| | | Adenosine/ | Wilcoxon |
|---|---|---|---|
| | Adenosine alone | dipyridamole | signed-rank test |
| Chest pain | 0.99 ± 1.93 | 0.37 ± 1.12 | 0.008 |
| Dyspnea | 2.55 ± 2.71 | 1.92 ± 2.34 | 0.02 |
| Flushing | 2.52 ± 2.63 | 1.64 ± 2.29 | 0.02 |
| Composite index* | 6.06 ± 3.17 | 3.94 ± 3.18 | <.0001 |

Composite index = Chest pain + dyspnea + flushing

Chest pain, dyspnea, flushing and the composite index (chest pain, dyspnea and flushing) were less severe in the adenosine/dipyridamole treatment group than in the Adenosine alone treatment group (p values of 0.008, 0.02, 0.02 and <0.0001, respectively).

As shown in Table 7 the number (and frequency) of adverse events related to the stimulation of A1 receptors, mainly chest pain, was reduced by 44% and its severity (Table 8 and 9) decreased by over 60% with the sequential combination treatment as compared to adenosine alone. The number (and frequency) of adverse events related to the stimulation of A2a receptors, mainly dyspnea and flushing, did not decrease. However their severity (not shown) decreased by 24 and 38% respectively, with the sequential combination compared to adenosine alone.

The mean coronary flow reserve (ratio of maximal-stimulated coronary blood flow “CBF” to baseline-resting CBF equivalent to peak and mean blood flow velocities ratios) of the 40 patients enrolled in the study was above 2 (normal value), which indicates that the observed reduction in side effects with sequential treatment was drug dependent, and not flawed by the ischemic status of the studied population.

Although not shown in the tabular data, EKG was not significantly different with the sequential combination treatment as compared to standard single-agent adenosine, and remained unchanged in all the patients. Vital signs (heart rate, systolic and diastolic blood pressures) changed similarly with the two methods. However, the heart rate increase and blood pressures decreases were less pronounced with the sequential combination than with adenosine alone.

It should be noted that in addition of the first series of 30 patients (DIP5 group), three (3) patients received the adenosine infusion 2 minutes after the dipyridamole 28 μg/kg bolus, and two (2) patients were injected the two agents (dipyridamole and adenosine) concurrently in the same infusion line using a “Y” connector using the same dose. These two modalities appeared equally effective and as effective as the immediate sequential administration protocol.

The results observed and protocol design were validated by two consecutive infusions of adenosine given at the standard dose of 140 mcg/kg/min and separated by a 5 minute interval: no difference was seen (p>0.05) in velocity measurements between the two infusion which confirms the documented fact that adenosine administered acutely and repetitively does not induce tachyphylaxis.

Results are shown in Table 10. Abbreviations used in the table are defined below:

PV: peak velocity (cm/sec)

MV: mean velocity (cm/sec)

max: velocity under stress conditions

min: velocity at rest (under basal conditions)

ADE1: first adenosine infusion

ADE2: second adenosine infusion

TABLE 10

| | PV- | | PV- | | MV- | | | |
|---|---|---|---|---|---|---|---|---|
| | ADE1 | | ADE2 | | ADE1 | | MV-ADE2 | |
| Patient ID | Max | Min | Max | Min | Max | Min | Max | Min |
| MART | 68 | 34 | 60 | 31 | 49 | 25 | 45 | 23 |
| LIEN | 65 | 23 | 66 | 22 | 48 | 17 | 48 | 16 |
| GRAND | 102 | 30 | 103 | 29 | 75 | 22 | 75 | 22 |
| NGHI | 73 | 30 | 63 | 26 | 53 | 23 | 47 | 21 |
| CORD | 70 | 28 | 62 | 25 | 50 | 20 | 48 | 20 |
| Mean | 75.6 | 29 | 70.8 | 26.6 | 55 | 21.4 | 52.6 | 20.4 |

| | | |
|---|---|---|
| Max | PV: 75.6 (ADE1) − | MV: 55 (ADE1) − |
| velocity | 70.8 (ADE2) = 4.8 | 52.6 (ADE2) = 2.4 |
| differ- | | |
| ential | | |
| P value | 0.12 | 0.11 |

In a second set of control experiments, dipyridamole was administered alone by bolus injection to 5 patients at the dosage of 28, 35 or 40 μg/kg after a three minute adenosine infusion at 140 μg/kg/min, and again after a 5 minute stabilization period. Data are shown in Table 11.

TABLE 11

| | DIP | PV- | | PV- | | MV- | | | | Symptoms | Symptoms |
|---|---|---|---|---|---|---|---|---|---|---|---|
| Patient | dose | ADE | | DIP | | ADE | | MV-DIP | | under | under |
| ID | (μg/kg) | Max | Min | Max | Min | Max | Min | Max | Min | ADE | DIP alone |
| GROS | 28 | 84 | 27 | 26 | 23 | 64 | 20 | 21 | 19 | Flushing | None |
| | | | | | | | | | | 8/10 | |
| COCH | 35 | 84 | 32 | 40 | 30 | 61 | 23 | 28 | 22 | Flushing | None |
| | | | | | | | | | | 6/10 | |
| WURI | 35 | 88 | 25 | 23 | 21 | 71 | 21 | 20 | 18 | None | None |
| STUR | 40 | 92 | 33 | 35 | 33 | 67 | 23 | 25 | 23 | Dyspnea | None |
| | | | | | | | | | | 5/10 | |
| TALL | 40 | 112 | 33 | 40 | 34 | 86 | 25 | 29 | 24 | None | None |

Dipyridamole alone did not modify peak and mean diastolic velocities. No symptoms were recorded during a follow-up of 10 minutes. The data demonstrate that intravenous bolus administration of dipyridamole as a single agent in the dosage range used in the experimental protocol, has no detectable effects; doses of dipyridamole at 28 to 40 μg/kg alone do not induce significant hemodynamic and clinical effects and therefore are subclinical doses.

### 5.2. Example 2

### Sequential and Concurrent Administration of the Combination. Single Photon Emission Computed Tomography (SPECT) Imaging Study

A Phase II study, now completed, compared dipyridamole-adenosine combination administration (also termed herein, at all doses, Adenosoft™) to adenosine alone (Adenoscan®, Astellas) as a pharmacologic stressor in coronary patients undergoing single photon emission computed tomography (SPECT) imaging studies. The study was a mono-center, single-blind, 2-arm, cross-over trial.

All patients underwent a first SPECT imaging study using adenosine as single agent pharmacologic stressor at 140 μg/kg/min, according to standard clinical protocol. Only those patients in whom an ischemic zone was detected were declared eligible for the second test, and were enrolled in the study if other inclusion criteria were satisfied.

In the second test, eligible patients were stressed pharmacologically by either bolus administration of dipyridamole over 20-30 seconds, followed by adenosine infusion at 70 μg/kg/min or their concurrent administration. SPECT images were acquired as per the standard approach performed the preceding week.

Since thallium is currently deemed the best isotope to test myocardial viability at rest, and sestamibi the best isotope to detect myocardial defects under stress conditions, a dual isotope myocardial scintigraphy technique was used for this study. The isotope was injected after a 3 minute infusion with Adenoscan or after a 2.5 minute infusion with the combination.

The primary end-point was to show the non inferiority of Adenosoft image vs. Adenoscan images. The comparison encompassed the strength of agreement for myocardial defects but also the severity of the reversible defects induced by the two products using a validated score method.

Randomization of SPECT images, and their analysis by two blinded readers, took place every 10 patients. Anonymous and randomized images were assessed using the standard 17-segment model and the semi-quantitative visual score method on a 5-point scale (from 0 to 4). An additional totally computerized quantitative method was performed using the validated CardioGam software (Segami Corp.). All images were interpreted and quantified automatically by the software calculation package.

Tolerance and safety (secondary end-point) of the procedure was analyzed as in the preceding hemodynamic study (Example 1) using a visual scale, focusing on the three most common symptoms seen with adenosine, as well as on EKG changes and other usual cardiac parameters.

5.2.1. SPECT Image Analysis and Scoring Method (Semi-Quantitative Analysis)

The “summed score” (SS) quantified the myocardial defect(s) in one or more of the 17 cardiac segments, as scored from 0 to 4 by the blinded readers using the following scale: normal perfusion=0, mild reduction in counts=1, moderate reduction in counts=2, severe reduction=3, absence of uptake=4.

Summed stress scores (with Adenosine or with Adenosoft) were defined as SSS, summed rest scores (Thallium) as SRS and summed difference scores as SDS=SSS-SRS. Delta scores were calculated as the difference between the summed difference scores of Adenoscan and the summed difference scores of the combination (Δ: delta scores=SDS (A)−SDS(C)), reflecting the difference between ischemia induced by adenosine and the combination. Ideally the delta scores of the two independent blind readers had not to differ by more than 2. If >2, images were read again by a third independent expert.

The severity and extent of reversible defects was categorized into mild (SDS 0 to 1), moderate (SDS 2 to 4) and severe (SDS >5).

Non inferiority of adenosine/dipyridamole compared to adenosine alone was performed using a unilateral paired t-test, taking α=0.05 and Δ=2 as the clinical bound under which the difference between the two treatments can be considered negligible. Concordance between the summed difference scores of the two methods was evaluated with the Kappa coefficient and the test of kappa (H0: Kappa coefficient=0.4).

There was a total of 56 patients included in the study however only 40 of them (who received the same total dose) were considered in the statistical analysis. The 16 patients who were not included in this analysis received doses of dipyridamole (28-35 μg/kg) in the combination that were less than the final dose (70 μg/kg/min+40 μg/kg dipyridamole combination) therefore it was not considered appropriate to include them in the statistical analysis.

The combination stressor of 40 μg/kg dipyridamole and 70 μg/kg/min adenosine given sequentially was tested and showed equivalent, and sometimes better results, than Adenoscan in terms of imaging efficacy in a series of ten consecutive patients Table 12 shows the stress scores (correlated to myocardial defects) for the first 10 patients of this 40 μg/kg series (see “Series I—sequential administration”).

TABLE 12

| | SDS.A | SDS.C | |
|---|---|---|---|
| Series I | (Adenoscan) | (SC) | Delta score |
| Patient 1 | 5 | 4 | 1 |
| Patient 2 | 13 | 11 | 2 |
| Patient 3 | 5 | 5 | 0 |
| Patient 4 | 4 | 4 | 0 |
| Patient 5 | 7 | 7.5 | −0.5 |
| Patient 6 | 5 | 5 | 0 |
| Patient 7 | 4 | 3 | 1 |
| Patient 8 | 14 | 14 | 0 |
| Patient 9 | 5 | 12.5 | −7.5 |
| Patient 10 | 5 | 6 | −1 |
| Total | | | −5 |
| Mean | | | −0.5 |
| Wilcoxon unilateral | | | P = 0.003 |
| (α = 0.05) | | | |

SDS = summed difference scores

A 100% agreement on myocardial defects summed stress scored ≧2 was found in this first series of 10 patients who received the combination sequentially (a 4 minute adenosine 70 μg/kg/min continuous IV infusion preceded by a bolus injection of dipyridamole 40 μg/kg) along with a negative total delta-score and a p value of 0.003 showing that the combination is non inferior to the reference drug.

A second series of 30 patients received the combination concurrently (adenosine 70 μg/kg/min with dipyridamole 10 μg/kg/min) for 4 minutes. Results are summarized in Table 13.

TABLE 13

| Concurrent administration-n = 30 pts | Delta score |
|---|---|
| Total | −6 |
| Mean | 0.2 |
| Unilateral paired T-test | p < 0.0001 |
| (α = 0.05) | |

Unilateral paired t-test showed a p value <0.0001 for both the concurrent administration group (n=30) and the total sample (n=40), thereby demonstrating that Adenosoft (adenosine/dipyridamole) is non inferior to Adenoscan (adenosine alone). The agreement rate for the severity of reversible defects based on an absolute difference ≦2 between the two summed stress scores was 73.3%. Concordance using 5 (SDS ≦5 or >5) as the threshold between severe and not severe reversible defects provided a kappa coefficient of 0.74 [0.50-0.97] for the concurrent administration group (n=30).

A computerized quantification of Summed Stress Scores and resulting delta scores was also performed and confirmed that there is no significant difference between Adenoscan and Adenosoft. Using this approach Mean delta score value was of −1 in the n=10 sequential group (p value=0.004) and of 0.36 in the n=30 concurrent group (p<0.0001).

With respect to patient tolerance in the largest series (n=30-concurrent administration): The occurrence of A2 symptoms with Adenosoft (as compared to Adenoscan) was reduced by 23% for dyspnea and 24% for flushing (not statistically significant). That of headache and of gastrointestinal discomfort was reduced by 20% and 45%, respectively (not statistically significant due to the low number of patients with these symptoms). The reduction of the occurrence of chest pain (A1 side effect) was reduced by 43% (p=0.003).

The severity of dyspnea and flushing was reduced by 49% and 51% (p=0.01 and 0.03), that of headache and of gastrointestinal discomfort, by 18% (not statistically significant) and 62.5% (p=0.06, close to statistical significance), respectively. The reduction of chest pain severity was reduced by 61% (p=0.001).

The summed score of the three main adverse symptoms forming the composite index was reduced by 53% with the combination compared to adenosine alone. This decrease was statistically significant (p<0.0001).

No bronchospasm was observed with either drug in this study.

There were no serious adverse effects or any event requiring hospitalization.

EKG changes: ST-T changes occurrence rate was significantly reduced by 67% with Adenosoft, as compared to Adenoscan in the total sample (n=40) and by 62% in the concurrent administration group alone. This difference was significant when considering the total sample (p=0.03) but not when considering the concurrent administration group alone (p=0.06) due to the lower number of patients. One AV-block of second grade was observed with the two compounds in the same patient, but the number of episodes was higher with Adenoscan than with Adenosoft.

Hemodynamic responses: No statistically significant difference was observed between the two compounds regarding blood pressure. However, heart rate was significantly lower (p=0.0005) during infusion with Adenosoft. Within 3 to 4 minutes after the end of the perfusion, both heart rate and blood pressure were at similar levels (lower HR with Adenosoft on average) with no statistical difference, suggesting a similar PK pattern for the two products.

Tolerance in the sequential administration (with dipyridamole 40 μg/kg immediately followed by adenosine 70 μg/kg/min) showed a clear trend towards a reduction of the severity of side effects in the combination group versus Adenoscan. However the difference did not reach statistical significance due to the small sample size studied (n=10).

Conclusion: Adenosoft is better tolerated by patients than Adenoscan. Most important side effects are significantly reduced in severity and occurrence with the combination of adenosine and dipyridamole.

### 5.3. Example 3

### Concurrent Administration-Reduction of Infusion Time-Assessment of Coronary Reserve by Transthoracic Doppler-Echo Study

An additional study of concurrent administration of dipyridamole 10 μg/kg/min with adenosine 70 μg/kg/min for two minutes was also performed using trans-thoracic doppler echography. The study compared the combination of adenosine and dipyridamole to adenosine alone given at its standard dosage (140 mg/kg/min) during the same period of time in 31 coronary patients assessed for coronary arterial flow reserve. The protocol was similar to that described in Example 1 for the sequential administration study: adenosine alone was infused first followed by a 5 minute interval at baseline and by the infusion of the combination thereafter.

Primary efficacy end-points were peak and mean diastolic flow velocities (their measurements reflect coronary blood flow values). The secondary end-point was patient's tolerance to the procedure.

Again results showed that the combination was as effective as adenosine with no statistical difference between the two methods (p>0.05) for both mean and peak velocities but significantly better tolerated. Results are summarized in Table 14 to 16.

TABLE 14

| | | Adenosine/ | |
|---|---|---|---|
| | Adenosine alone | dipyridamole | P Value |
| Mean velocity at baseline | 25.2 ± 8.9 | 25.5 ± 9.6 | 0.25 |
| Peak velocity at baseline | 33.0 ± 12.9 | 33.8 ± 14.2 | 0.20 |
| Delta mean velocity | 41.5 ± 15.3 | 42.5 ± 15.2 | 0.50 |
| Delta peak velocity | 54.4 ± 20.5 | 56.6 ± 21.1 | 0.26 |

“MV-BL” = mean velocity at rest (baseline) -
“PV-BL” = peak velocity at rest (baseline) -
“MV-STR” = mean velocity under stressing agent infusion -
“PV-STR” = peak velocity under stressing agent infusion.
Delta = peak-BL.

Results also demonstrated that coronary reserve assessments are the same with the two products showing no statistical difference between them (P>0.05).

TABLE 15

| | | Adenosine/ | |
|---|---|---|---|
| | Adenosine alone | dipyridamole | P Value |
| Coronary reserve assessment | 2.79 ± 0.90 | 2.79 ± 0.86 | 0.98 |
| using mean velocity | | | |
| Coronary reserve assessment | 2.82 ± 0.96 | 2.85 ± 0.94 | 0.64 |
| using peak velocity | | | |

With respect to tolerance there was no clear difference in this series regarding the occurrence of adverse events between the two products. However a significant difference in the severity of symptoms was again observed as can be seen in Table 16 with further decrease of A2 side effects severity [dyspnea by 33% (p=0.001) and flushing by 77% (p=0.008)] by comparison to the first TTDE study results described in Example 1. This is likely to be due to the reduction in infusion time to 2 minutes instead of 3 minutes as in the previous TTDE study.

Table 16. Severity of the main symptoms (fixed concurrent dose adenosine 70 μg/kg/min+dipyridamole 10 Ξg/kg/min vs. adenosine 140 μg/kg/min).

TABLE 16

| | Adenosine alone | Adenosine/dipyridamole | P |
|---|---|---|---|
| Chest pain | 0.68 ± 1.87 | 0.23 ± 0.96 | 0.12 |
| Dyspnea | 3.64 ± 3.67 | 2.39 ± 3.06 | 0.001 |
| Flushing | 4.50 ± 3.15 | 3.00 ± 2.67 | 0.008 |
| Composite index* | 8.82 ± 5.62 | 5.61 ± 4.62 | 0.0002 |

Composite index = Chest pain, dyspnea and flushing
Only 4 patients in the adenosine group and 2 patients in the combination group experienced thoracic discomfort in this series explaining the lack of statistical significance for this symptom in the combination treated group versus adenosine alone treated subjects.

### 5.4. Example 4

### Priming Dose Effect

Combination treatment remained equivalent to treatment with the reference drug when infusion time was reduced to 2 minutes from 3 minutes as described in Example 1. Reducing the time of infusion (from 3 to 2 minutes for TTDE studies and from 4 to 3 minutes for SPECT studies and possibly less) can further reduce the incidence and severity of side effects.

It is known that, in addition to being absorbed by red blood cells, adenosine is taken up and eliminated by the lungs; inhibition of this elimination by dipyridamole produces an elevated concentration of endogenous adenosine in the heart; and the dipyridamole effect occurs rapidly (e.g in less than ten seconds at 0° Celsius in human erythrocytes). At 37° Celsius the dipyridamole inhibitory effect on the capture of adenosine by red blood cells, platelets and endothelial cells (mainly at the pulmonary capillary bed level) is presumed to take longer time than at low temperatures because adenosine uptake velocity is then increased about three fold. The study results described in Example 3 and with a total infusion time of 2 minutes and measurements performed between 45 to 90 seconds, suggests that dipyridamole effect becomes effective after a very short period of time in the order of no more than two to three dozen of seconds. In the guinea-pig, whose sensitivity to dipyridamole is very close to that of human, the ¹⁴C-content of the lungs following an intravenous injection of ¹⁴C-adenosine, is lowered after only 20 seconds by low doses of dipyridamole while that of the heart is increased (see Table 17 below—Kolassa & al., European J Pharmacology, 1971,13,320,-325).

TABLE 17

| Pretreatment | Lungs | Heart |
|---|---|---|
| Controls (n = 5) | 81 ± 5.5 | 1.7 ± 0.6 |
| Dipyridamole | | |
| 0.025 mg (n = 5) | 71.4 ± 4.9 (p < 0.05) | 2.1 ± 1.01 |

¹⁴C distribution after ¹⁴C-adenosine intravenous injection-Mean values as a percentage of ¹⁴C administered

As a test, a low priming dose of dipyridamole was combined with the concurrent administration of the two drugs to determine whether the time to peak and the occurrence of near maximal hyperaemia could be accelerated, especially when infusion time is reduced by, for example, one minute.

I assessed the effects of several dipyridamole priming doses by measuring time to peak (as reflecting differences in adenosine concentration in the heart) in a series of patients with normal coronary reserve. Patients were divided into 4 parallel groups (n=3 to 6) who received a 3 minute infusion of adenosine alone at 140 μg/kg/min, followed by a 5 minute interval at baseline, and thereafter a concurrent administration of 70 μg/kg/min adenosine with 10 μg/kg/min dipyridamole, this concurrent administration performed according to three different protocols: (i) without a dipyridamole priming dose (n=6) or (ii) preceded by a priming dose (n=12) of either 0.05 mg, 0.1 or 0.3 mg (representing a fraction of the total dose) injected via a Y connector into the infusion line.

In 3 consecutive patients a dipyridamole priming dose of 0.05 mg did not modify time to peak, so this dose was not explored further. In 3 other consecutive patients, a priming dose of 0.1 mg did modify this parameter (see Table 17 and 18) so that a total of 6 patients were studied at this dose. Three more patients received a priming dose of 0.3 mg that did not perform better than 0.1 mg, so that no additional subjects were explored at this dose

TABLE 18

| | | Concurrent administration without |
|---|---|---|
| N = 6 PTS | ADE | dipyridamole priming dose |
| Time is | [140 μg/ | C [Dip 10 μg/kg/min + Ade |
| expressed in seconds | kg/min] | 70 μg/kg/min] |
| Time to peak pt 1 | 85 | 90 |
| Time to peak pt 2 | 80 | 110 |
| Time to peak pt 3 | 75 | 85 |
| Time to peak pt 4 | 56 | 58 |
| Time to peak pt 5 | 120 | 130 |
| Time to peak pt 6 | 50 | 60 |
| Total | 416 | 533 |
| Mean | 69.3 | 88.8 |
| Return to BL pt 1 | 75 | 100 |
| Return to BL pt 2 | 60 | 60 |
| Return to BL pt 3 | 100 | 111 |
| Return to BL pt 4 | 48 | 63 |
| Return to BL pt 5 | 60 | 55 |
| Return to BL pt 6 | 60 | 79 |
| Total | 403 | 468 |
| Mean | 67.1 | 78 |
| N = 6 PTS | | Concurrent administration with |
| Time is | | dipyridamole priming dose (0.1 mg) |
| expressed in seconds | ADE | C |
| Time to peak pt 1 | 74 | 60 |
| Time to peak pt 2 | 55 | 70 |
| Time to peak pt 3 | 45 | 60 |
| Time to peak pt 4 | 44 | 46 |
| Time to peak pt 5 | 30 | 35 |
| Time to peak pt 6 | 40 | 42 |
| Total | 288 | 313 |
| Mean | 48 | 52.1 |
| Return to BL pt 1 | 55 | 80 |
| Return to BL pt 2 | 65 | 85 |
| Return to BL pt 3 | 54 | 60 |
| Return to BL pt 4 | 67 | 60 |
| Return to BL pt 5 | 48 | 60 |
| Return to BL pt 6 | 60 | 65 |
| Total | 349 | 350 |
| Mean | 58.1 | 58.3 |

Results showed that time to peak with the combination is, on average, 1.5 minutes versus about 1 minute with adenosine, but approaches that of adenosine alone (with no change in return to baseline time) when a dipyridamole priming dose is administered prior to the concurrent infusion of the combination. The shortest time to peak being most appropriate to secure the efficacy of the combination given concurrently, this approach appeared to be an improvement of the method whatever the total infusion duration. A priming dose of dipyridamole (given prior to the concurrent infusion) and acting as pre-treatment (presumably, but not intending to be bound by theory, by partial inhibition of adenosine capture by pulmonary endothelial cells) can achieve such effect. With regard to tolerance, this method of use can help further reduce the total infusion time, thus the total dose received by the patient and along with that the occurrence and severity of related side effects

These data suggest that a 3 minute infusion instead of a 4 minute infusion could be possibly used for SPECT studies with injection of the isotope after about only 2 minutes. Further, the data suggest that a 2 minute infusion instead of a 3 to 4 minute infusion could be used for MCE studies, and especially CT studies, which have the added advantage of being performed without administration of contrast agent.

### 5.5. Example 5

### Priming Dose Effect with the Sequential Administration Mode

The priming dose effect was also studied in 10 consecutive patients who were administered the combination sequentially (dipyridamole 40 μg/kg as a 5-10 seconds bolus, followed by adenosine infusion 70 μg/kg/min over 2 minutes). Again there was no difference between the two methods for both peak and mean velocities. Results show that time to peak and return to baseline are almost the same between the two method (see Table 19).

TABLE 19

| N = 10 PTS | Adenosine alone | Sequential administration |
|---|---|---|
| Time is | ADE [140 | C [Dip 40 μg/kg + Ade |
| expressed in seconds | μg/kg/min] | 70 μg/kg/min] |
| Time to peak pt 1 | 60 | 60 |
| Time to peak pt 2 | 90 | 140 |
| Time to peak pt 3 | 40 | 60 |
| Time to peak pt 4 | 50 | 60 |
| Time to peak pt 5 | 70 | 75 |
| Time to peak pt 6 | 50 | 60 |
| Time to peak pt 7 | 75 | 80 |
| Time to peak pt 8 | 60 | 45 |
| Time to peak pt 9 | 35 | 35 |
| Time to peak pt 10 | 38 | 36 |
| Total | 568 | 651 |
| Mean | 57 | 65 |
| Return to BL pt 1 | 30 | 50 |
| Return to BL pt 2 | 30 | 75 |
| Return to BL pt 3 | 60 | 65 |
| Return to BL pt 4 | 50 | 30 |
| Return to BL pt 5 | 60 | 70 |
| Return to BL pt 6 | 60 | 80 |
| Return to BL pt 7 | 35 | 40 |
| Return to BL pt 8 | 40 | 40 |
| Return to BL pt 9 | 35 | 25 |
| Return to BL pt 10 | 28 | 37 |
| Total | 428 | 512 |
| Mean | 43 | 51 |

### 5.6. Example 6

### Stability of a Single Unit Dosage Form Comprising Adenosine and Dipyridamole in the 7:1 Ratio

A study was undertaken to assess pharmacological properties of a pharmaceutical composition comprising adenosine and dipyridamole at an A:D weight ratio of 7:1. In particular, the study was conducted to assess the properties of a composition comprising adenosine at a concentration of 7 mg/ml and dipyridamole at a concentration of 1 mg/ml. This particular formulation is very convenient, since it permits easy calculation of concentrations, maximal volumes, and weights—which is useful to reduce dosing errors in the clinical setting—while also covering a wide range of needs, as shown in the Table 20 below

TABLE 20

| | | | Maximal | Total |
|---|---|---|---|---|
| Adenosine | | Total amount | patient | amount of |
| concentration | Volume | of adenosine | weight covered | dipyridamole |
| (mg/ml) | (ml) | per unit (mg) | (kgs) | per unit (mg) |
| 7 | 2 | 14 | 50 | 2 |
| | 3 | 21 | 75 | 3 |
| | 4 | 28 | 100 | 4 |
| | 5 | 35 | 125 | 5 |
| | 6 | 42 | 150 | 6 |
| | 8 | 56 | 200 | 8 |

Dipyridamole is poorly soluble in saline and unstable in the long term in solvents at pH>4. Adenosine compositions currently used in clinical practice have pH>4, and are thus poorly suited to addition of dipyridamole. Therefore, a more acidic pH was chosen. The lower pH also increases adenosine solubility above 4 mg/ml, which is the upper acceptable adenosine concentration limit in saline.

The following composition (Table 21) was prepared:

TABLE 21

| | Final | | |
|---|---|---|---|
| Component | concentration | Supplier | Reference |
| Adenosine | 7 mg/ml | Sigma | Eur.Ph. 01/2005: 1486 |
| | | Aldrich | |
| Dipyridamole | 1 mg/ml | Sigma | Eur.Ph. 01/2005: 1199 |
| | | Aldrich | |
| Polyethyleneglycol | 50 mg/ml | SASOL | Eur.Ph. 01/2005: 1444 |
| 600 | | | |
| Tartaric acid | 2 mg/ml | Sigma | Eur.Ph. 01/2005: 0460 |
| | | Aldrich | |
| Water for injection | — (1 ml) | Aguettant | AMM: 319 508.5 |

After sonication for 2 minutes and magnetic stirring for 10 minutes, the solution became completely clear with a pH of 3.6 and an osmolality of 151 mosmol/kg. The stability of this composition was studied at various temperatures and conditions over a 6 month period of time. Results are summarized in the Table 22 below.

TABLE 22

| Controls | Specifications | T0 | T3 months | T6 months |
|---|---|---|---|---|
| | | Storage conditions: | | |
| | | 25° C. ± 2° C.-60% | | |
| | | HR ± 5% - Results | | |
| Characters | | | | |
| Appearance | Yellowish clear | complies | complies | complies |
| | solution | | | |
| pH (3.4) | 2.5-4.5 | 3.6 | 3.7 | 3.6 |
| Osmolality | 130-180 mosmol/kg | 153 | 152 | 147 |
| Identifications | | | | |
| Adenosine by HPLC | The retention time and | complies | complies | complies |
| Dipyridamole by HPLC | the UV spectra | complies | complies | complies |
| | are the same as the | | | |
| | standard solution for the | | | |
| | two products | | | |
| Assays | | | | |
| Adenosine by HPLC | 0.665-0.735 g/100 ml | 0.693 | 0.686 | 0.718 |
| Dipyridamole by | 0.095-0.105 g/100 ml | 0.101 | 0.097 | 0.094 |
| HPLC | | | | |
| | | Storage conditions: | | |
| | | 5° C. ± 3° C. - Results | | |
| Characters | | | | |
| Appearance | Yellowish clear | | | |
| | solution | | | |
| pH (3.4) | 2.5-4.5 | | | |
| Osmolality | 130-180 mosmol/kg | | | |
| Identifications | | | | |
| Adenosine by HPLC | The retention time | complies | complies | complies |
| Dipyridamole by | and the UV spectra | complies | complies | complies |
| HPLC | are the same as the | | | |
| | standard solution for | | | |
| | the two products | | | |
| Assays | | | | |
| Adenosine by HPLC | 0.665-0.735 g/100 ml | 0.710 | 0.707 | 0.693 |
| Dipyridamole by HP | 0.095-0.105 g/100 ml | 0.098 | 0.098 | 0.099 |

At 30° C. and above (not shown) the stability of the composition appeared below acceptable specifications after 3 months. At 25° C. and a relative humidity of 60% it appeared slightly under the required specifications (table 22) however some mild modifications brought to the original medium could normalized this result according to known art in this field. At storage conditions of 5° C. and after 6 months the composition as described here appeared perfectly stable and within usual specifications required.

### 5.7. Example 7

### Specific Labels with Kilogram Graduation Marks for Syringes

A label for synthetic (plastic, resin, polymer, or equivalent) or glass syringes prefilled with 1 ml of dipyridamole alone (typically 4 mg) with kilo graduation marks on the barrel is designed according to the following recommendations: the label provides 0.01 ml intervals per kilogram such that 1 ml corresponds to 100 kg of patient weight, 0.9 ml corresponds to 90 kg, 0.8 ml corresponds to 80 kg and so forth. See, e.g., FIG. 2A. Scales and numbers are inscribed on the cylinder (barrel). Mid values (e.g., 95, 85, 75 kg or 0.95, 0.85 or 0.75 ml) are marked by a longer graduation mark, typically about twice the length of the smallest increment on the label (such as, for example 82, 73 or 61 kg or 0.82, 0.73 or 0.61 ml). Labels may comprise a scale ranging from 10 to 100 kilograms or a more limited scale from 100 to 40 kilos (see, e.g., FIGS. 1A-1B). Labels for larger volume prefilled syringes are also possible. For example, labels are feasible for a 2 ml prefilled dipyridamole syringe using the same scale as above but twice as long and ranging up to 200 kilograms.

A label for synthetic (plastic, resin, polymer or equivalent) or glass syringes filled with 10 ml of adenosine alone (typically 28 mg) is designed according to the same principle as in the immediately preceding paragraph with several possible intervals depending on the infusion time. On one label, 1 kilogram is equivalent to 0.1 ml (infusion time=4 minutes). On another label 1 kilogram is equivalent to 0.075 ml (infusion time=3 minutes). See, e.g., FIG. 2B. On another label (30 mg adenosine in 10 ml and infusion time 3 minutes), 1 kilogram is equivalent to 0.07 ml.

Labeled pre-filled syringes with joined (yoked) plungers to allow for coordinated administration of adenosine and dipyridamole are also envisioned. See, e.g., FIG. 6. For example, a 1-ml prefilled dipyridamole syringe and a 10 ml prefilled adenosine syringe can be adjusted in order to be of the same length, and then easily joined by known means, so that the motion of these two pistons is coordinated. At the two open ends of two syringes a connector can be either pushed or screwed to facilitate the mixing of the two products.

### 5.8. Example 8

### Labels with Kilogram Graduation Marks for Syringes

In some label embodiments, the weight graduation scale is in kilograms and the scale can range from 10 or 40 kilograms up to 100 or 125 or 133 or 150 or 166 or even 200 kilograms depending on the syringe capacity. See FIG. 1. Milliliter per kilogram equivalence can range from 1 kilogram=0.0005 ml, to 1 kilogram=1 ml regardless of the syringe characteristics and more typically from 1 kilogram=0.01 ml, to 1 kilogram=0.2 ml.

From the description herein, labels can be readily designed for syringes of varying sizes and volumes. Table 23 below provides examples of three different possible kilogram scales for prefilled syringes ranging from 0.1 to 10 ml.

TABLE 23

| | Kilogram graduation | | |
|---|---|---|---|
| | interval in ml (One | | |
| Prefilled syringe | kilogram/value in ml) | | |
| (in milliliters) ml | 50 kg | 100 kg | 125 kg |
| 0.1 | 0.002 | 0.001 | 0.0008 |
| 0.2 | 0.004 | 0.002 | 0.0016 |
| 0.3 | 0.006 | 0.003 | 0.0024 |
| 0.4 | 0.008 | 0.004 | 0.0032 |
| 0.5 | 0.010 | 0.005 | 0.0040 |
| 0.6 | 0.012 | 0.006 | 0.0048 |
| 0.7 | 0.014 | 0.007 | 0.0056 |
| 0.8 | 0.016 | 0.008 | 0.0064 |
| 0.9 | 0.018 | 0.009 | 0.0072 |
| 1 | 0.02 | 0.010 | 0.0080 |
| 1.5 | 0.03 | 0.015 | 0.012 |
| 2 | 0.04 | 0.020 | 0.016 |
| 2.5 | 0.05 | 0.025 | 0.020 |
| 3 | 0.06 | 0.030 | 0.024 |
| 3.5 | 0.07 | 0.035 | 0.028 |
| 4 | 0.08 | 0.040 | 0.032 |
| 4.5 | 0.09 | 0.045 | 0.036 |
| 5 | 0.10 | 0.050 | 0.040 |
| 5.5 | 0.11 | 0.055 | 0.044 |
| 6 | 0.12 | 0.060 | 0.048 |
| 6.5 | 0.13 | 0.065 | 0.052 |
| 7 | 0.14 | 0.070 | 0.056 |
| 7.5 | 0.15 | 0.075 | 0.060 |
| 8 | 0.16 | 0.08 | 0.064 |
| 8.5 | 0.17 | 0.085 | 0.068 |
| 9 | 0.18 | 0.09 | 0.072 |
| 9.5 | 0.19 | 0.095 | 0.076 |
| 10 | 0.20 | 0.1 | 0.080 |

If the label ranges up to 200 kgs, a 1-kg interval is equivalent to (0.1 ml/200) or 0.0005 ml. As shown here, for a 10-ml syringe, the greatest ml per kg division is 0.2 ml. Similarly, for 20 or 30 ml prefilled syringes, the greatest ml per kg division is 0.4 ml or 0.6 ml respectively. Incremental values of ml per kilo can range up to 0.8 ml/kg, or even 1 ml/kg.

Labels can be tailored for any desired starting and ending weight. Examples of upper ends of weight scales include, but are not limited to 200 kg, 150 kg, or 120 kg. In some cases, the ml per kilo equivalence is fractional rather than a whole number, such as 0.000666 (0.1 ml/150 kg) or 0.006 (1 ml/150 kg) or 0.0008333 (0.1 ml/120 kg) or 0.008333 (1 ml/120 kg).

Labels can also be designed for low weight patients (e.g., children) as shown in the 50 kg example above. Equally possible are labels with weight scales ranging up to a maximum of 40 kg or even 30 kg.

In some embodiments, the weight scale is labeled in pounds and the unit interval is one pound or two pounds corresponding to a certain volume of active solution, following the same principle as described for kilograms.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

1. A pharmaceutical composition comprising adenosine and dipyridamole in an adenosine:dipyridamole weight ratio of about 2:1 to about 10:1.

2. (canceled)

3. The pharmaceutical composition according to claim 1, wherein adenosine and dipyridamole are present in amounts that permit adenosine to be administered at a dosage rate of 35 to 100 μg/kg/min and dipyridamole to be administered at a dosage rate of 3.5 to 50 μg/kg/min.

4. The pharmaceutical composition of claim 3, wherein the composition is a sterile fluid.

5. The pharmaceutical composition of claim 4, wherein adenosine and dipyridamole are present at concentrations that permit direct intravenous administration.

6. (canceled)

7. The pharmaceutical composition according to claim 1, wherein the concentration of adenosine is about 1 to 10 mg/ml.

8. (canceled)

9. The pharmaceutical composition according to claim 1, wherein the concentration of dipyridamole is about 0.1 to 4 mg/ml.

10. The pharmaceutical composition according to claim 9, wherein the concentration of dipyridamole is about 1 mg/ml and the concentration of adenosine is about 7 mg/ml.

11. A unit dosage form comprising about 2 to 50 ml of the composition according to claim 1, wherein the composition is a sterile fluid.

12-18. (canceled)

19. A unit dosage form comprising about 5 to 60 mg of adenosine and about 0.1 to 10 mg of dipyridamole, wherein the composition is a solid capable of sterile reconstitution in a physiologically acceptable solvent or solution.

20-24. (canceled)

25. A unit dosage form of dipyridamole, comprising dipyridamole at a concentration of 0.1 to 4 mg/ml.

26-30. (canceled)

31. A unit dosage form of adenosine, formulated in sterile fluid composition, wherein the dose packaging permits sterile introduction of a second fluid in a volume at least 15% that of the adenosine composition.

32. The unit dosage form of claim 31, comprising 21 mg adenosine in 6 ml.

33. The unit dosage form of claim 31, comprising 28 mg adenosine in 6 ml.

34. The unit dosage form of claim 31, comprising 42 mg adenosine in 12 ml.

35. The unit dosage form of claim 31, comprising 56 mg adenosine in 12 ml.

36. The unit dosage form of claim 31, comprising 35 mg adenosine in 14 ml.

37. A unit dosage form of adenosine, formulated in sterile fluid composition, comprising adenosine at a concentration of about 2 to about 4 mg/ml.

38. (canceled)

39. The unit dosage form of claim 37, comprising 21 mg adenosine in 7 ml.

40. The unit dosage form of claim 37, comprising 42 mg adenosine in 14 ml.

41. The unit dosage form of claim 38, comprising 28 mg adenosine in 7 ml.

42. The unit dosage form of claim 38 comprising 56 mg in 14 ml.

43. A unit dosage form of adenosine, formulated in sterile fluid composition, comprising adenosine at a concentration of about 2.1 mg/ml.

44. A unit dosage form of adenosine, formulated in sterile fluid composition, comprising adenosine at a concentration of about 2.8 mg/ml.

45-48. (canceled)

49. A label for dose adjustment based on patient weight, the label capable of being affixed to a delivery device and comprising at least one graduated scale in units of weight.

50. The label according to claim 49, comprising one or more graduation scales permitting the dosing of dipyridamole alone, adenosine alone, or a composition comprising adenosine and dipyridamole.

51. The label of claim 50, affixed to a prefilled syringe comprising a pharmaceutical composition of adenosine alone, dipyridamole alone, or both in combination.

52. The label of claim 51 for prefilled syringes with dipyridamole alone wherein a 1 kilogram interval is equivalent to 0.01 ml.

53. The label of claim 49 for prefilled syringes comprising a solution of adenosine alone wherein a one kilogram interval on the scale is equivalent to 0.06 ml, 0.07 ml, 0.075 ml, 0.08 ml, 0.09 ml, 0.1 ml or 0.12 ml.

54. A kit comprising at least one unit dosage form of dipyridamole and at least one unit dosage form of adenosine.

55-56. (canceled)

57. A method of effecting coronary vasodilation for cardiac diagnosis, the method comprising: concurrently administering adenosine and dipyridamole, from a single unit dosage form or from separate unit dosage forms, optionally with a dipyridamole priming dose prior to their concurrent infusion, and wherein adenosine and dipyridamole are administered parenterally at an adenosine:dipyridamole weight ratio of about 2:1 to about 10:1.

58. (canceled)

59. The method of claim 57, wherein adenosine is administered at a dosage rate of 35 to 100 μg/kg/min and dipyridamole is administered at a dosage rate of 3.5 to 50 μg/kg/min.

60. The method of claim 57 wherein adenosine is administered at a dosage rate of 70 μg/kg/min and dipyridamole is administered at a dosage rate of 10 μg/kg/min.

61. The method of claim 57 wherein a dipyridamole priming dose of about 0.05 to 0.5 mg is administered prior to concurrent administration of adenosine with dipyridamole.

62. (canceled)

63. The method of claim 57 wherein adenosine and dipyridamole are administered concurrently from separate unit dosage forms and compositions.

64. The method of claim 63 wherein adenosine and dipyridamole are administered concurrently from separate, interconnected syringes.

65. The method of claim 57, wherein adenosine and dipyridamole are parenterally administered continuously for a period of about 1 to 6 minutes.

66-69. (canceled)

70. The method of claim 57, wherein the adenosine and dipyridamole compositions are administered by intravenous infusion.

71. The method of claim 57, wherein the adenosine and dipyridamole compositions are administered intra-arterially.

72. The method of claim 57, further comprising the step of assessing cardiac function.

73. The method of claim 72, wherein assessing cardiac function includes use of one or more techniques selected from the group consisting of: electrocardiography, M mode echography, two dimensional echography, three dimensional echography, echo-doppler, cardiac imaging, planar (conventional) scintigraphy, single photon emission computed tomography (SPECT), dynamic single photon emission computed tomography, positron emission tomography (PET), first pass radionuclide angiography, equilibrium radionuclide angiography, nuclear magnetic resonance (NMR) imaging, myocardial perfusion contrast echocardiography, digital subtraction angiography (DSA), x-ray computed tomography (CINE CT), high speed and ultra high speed computed tomography scan.

75-78. (canceled)

79. The method of any one of claim 72, wherein cardiac function assessment includes parenteral administration of an isotope, and wherein the isotope is administered after 2 minutes and before 3 minutes from the start of the concurrent parenteral administration of adenosine and dipyridamole.

80. (canceled)

81. A method of effecting coronary vasodilation for cardiac diagnosis, the method comprising parenterally administering dipyridamole as a pretreatment; and sequentially thereafter parenterally administering an adenosine receptor agonist,
wherein each of dipyridamole and said adenosine receptor agonist is administered at a dosage lower than that required for maximal coronary vasodilation when administered as a single agent by identical parenteral route.

82. The method of claim 81, wherein the adenosine receptor agonist is selected from the group consisting of: adenosine, adenosine triphosphate, adenosine diphosphate, adenosine monophosphate, and pro-drugs and pharmaceutically acceptable salts thereof and wherein the adenosine agonist: dipyridamole ratio is of about 2:1 to about 10:1.

83. The method of claim 81, wherein each route of parenteral administration is independently selected from the group consisting of: intra-arterial, intravenous, intra-coronary, and atrial administration.

84. The method of claims 81, wherein dipyridamole is administered by intravenous bolus injection prior to adenosine agonist infusion.

85. The method of claim 81, wherein dipyridamole is administered as an intravenous or intra-arterial bolus at a dosage of 14 to 140 μg/kg and adenosine intracoronarily at the dose of 10-20 μg/min for up to a maximum of about 6 minutes.

86. (canceled)

87. The method of claim 81 wherein dipyridamole is administered intravenously as a bolus over 5 to 30 seconds.

88. The method of claim 81, wherein administration of the adenosine receptor agonist is begun immediately after completion of dipyridamole administration.

89. The method of claim 88, wherein adenosine receptor agonist is infused intravenously for about 1 to about 6 minutes after dipyridamole injection.

90-92. (canceled)

93. The method according to claim 81, wherein the adenosine receptor agonist is adenosine, administered by intravenous infusion at a dosage rate of 35 to 100 μg/kg/min.

94. The method of claim 93, wherein adenosine is administered at a dosage rate of about 70 μg/kg/min.

95. The method of claim 81, wherein dipyridamole is administered intravenously and adenosine is administered intravenously.

96. The method of claim 81 wherein the adenosine receptor agonist is adenosine, the total dose of dipyridamole is 23 to 40 μg/kg, and the dosage rate for adenosine is 50 to 70 μg/kg/min.

97. (canceled)

98. The method of claim 81, further comprising the step of: assessing cardiac function.

99. The method of claim 98, wherein the step of assessing cardiac function includes use of one or more techniques selected from the group consisting of: electrocardiography, M mode echography, two dimensional echography, three dimensional echography, echo-doppler, cardiac imaging, planar (conventional) scintigraphy, single photon emission computed tomography (SPECT), dynamic single photon emission computed tomography, positron emission tomography (PET), first pass radionuclide angiography, equilibrium radionuclide angiography, nuclear magnetic resonance (NMR) imaging, myocardial perfusion contrast echocardiography, digital subtraction angiography (DSA), x-ray computed tomography (CINE CT), high speed CT scan, and ultra high speed CT scan.

100-103. (canceled)

104. The method of claim 99 wherein the isotope is injected after 2 and before 3 minutes after the start of adenosine receptor agonist infusion.

105. The method of claim 99 wherein assessing cardiac function includes parenteral administration or formation of any agent detectable by ultrasound techniques into the coronary artery network.

106. The unit dosage form of claim 37, comprising 14 mg adenosine in 7 ml.

107. The label of claim 49, wherein the delivery device is a syringe.

108. The label of claim 107, wherein the syringe has a total capacity of 1 to 30 ml.

109. The label of claim 49, wherein the unit of weight is a kilogram.

110. The label of claim 109, wherein the scale ranges from a minimum of about 10-40 kg to a maximum of about 100-200 kg.

111. The label of claim 49, wherein the unit of weight is a pound.

112. The label of claim 49, further comprising a graduated scale in units of volume.

113. The label of claim 112, wherein a 1 kg interval corresponds to a volume ranging from 0.0005 ml to 1 ml.

114. The label according to claim 51, wherein the prefilled syringe contains a composition comprising 60 mg of adenosine in 20 ml in a prefilled syringe.

115. The label according to claim 51, wherein the prefilled syringe contains a composition comprising 90 mg of adenosine in 30 ml in a prefilled syringe.

116. The method of claim 72, wherein the cardiac function assessed is myocardial perfusion or vasodilation of a coronary artery.

117-119. (canceled)

120. The label of claim 49 for prefilled syringes comprising a solution of adenosine alone wherein a one kilogram interval on the scale is equivalent to 0.03 ml, 0.04 ml, 0.042 ml, 0.0525 ml, 0.056 ml, or 0.07 ml.
